(19) United States
(12) Patent Application Publication
(10) Pub. No.: US 2025/0090741 A1
(43) Pub. Date: Mar. 20, 2025

(54) SYSTEMS AND METHODS FOR DETECTING NEGATIVE PRESSURE WOUND THERAPY DRESSING LEAKAGE USING STETHOSCOPE

(71) Applicant: KCI Manufacturing Unlimited Company, Westmeath (IE)

(72) Inventors: Timothy M. ROBINSON, Shillingstone (GB); Benjamin A. PRATT, Poole (GB); Mike BEASLEY, Wimborne (GB); Richard M. KAZALA, Jr., San Antonio, TX (US)

(21) Appl. No.: 18/701,174

(22) PCT Filed: Sep. 26, 2022
(86) PCT No.: PCT/IB2022/059117

§ 371 Date: Apr. 12, 2024

Related U.S. Application Data
(60) Provisional application No. 63/255,107, filed on Oct. 13, 2021.

(51) Int. Cl. A61M 1/00

(52) U.S. Cl. CPC A61M 1/91; A61M 1/98; A61M 2202/0014; A61M 2202/04; A61M 2205/15; A61M 2205/3334; A61M 2205/3375; A61M 2205/3561; A61M 2205/3576; A61M 2205/502; A61M 2205/581; A61M 2205/583

(57) ABSTRACT

A method for checking a seal of a negative wound therapy (NPWT) dressing includes detecting a presence of a leak in a NPWT dressing using a NPWT device, displaying instructions to identify the leak using an electronic stethoscope via a user interface, and determining a location of the leak in the NPWT dressing using the electronic stethoscope.

### CROSS-REFERENCE TO RELATED APPLICATIONS

This application is a U.S. National Stage Entry of PCT/IB2022/059117, filed Sep. 26, 2022, which claims the benefit, under 35 U.S.C. § 119 (e), of U.S. Provisional Patent Application No. 63/255,107, entitled “SYSTEMS AND METHODS FOR DETECTING NEGATIVE PRESSURE WOUND THERAPY DRESSING LEAKAGE USING STETHOSCOPE,” filed Oct. 13, 2021, each of which are incorporated herein by reference for all purposes.

### BACKGROUND

The present disclosure relates generally to a wound therapy system, and more particularly to a negative pressure wound therapy system.

### SUMMARY

One implementation of the present disclosure is a method for checking a seal of a negative wound therapy (NPWT) dressing. The method includes detecting a presence of a leak in a NPWT dressing using a NPWT device, displaying instructions to identify the leak using an electronic stethoscope via a user interface, and determining a location of the leak in the NPWT dressing using the electronic stethoscope.

In some embodiments, the instructions to identify the leak using an electronic stethoscope include displaying a plurality of steps for performing by a user. In some embodiments, the plurality of steps comprise moving a transducer of the electronic stethoscope around the NPWT dressing.

In some embodiments, the plurality of steps includes steps for pairing the electronic stethoscope to at least one of the NPWT device or a user device.

In some embodiments, the user device is in communication with the NPWT device.

In some embodiments, the method includes measuring a characteristic of the leak using the electronic stethoscope.

In some embodiments, the method includes determining a flow rate of the leak based on the characteristic measured by the electronic stethoscope.

In some embodiments, the method includes displaying, via graphical user interface, the characteristic of the leak.

In some embodiments, the method includes indicating the characteristic of the leak via at least one of a light emitting device and a sound emitting device.

In some embodiments, the method includes determining a characteristic of the leak based on a static negative pressure applied to the NPWT dressing and based on a signal generated by a transducer of the electronic stethoscope.

In some embodiments, the negative pressure pump of the NPWT device is disabled while the signal generated by the transducer of the electronic stethoscope is obtained.

In some embodiments, the NPWT device includes the user interface. In some embodiments, the user interface includes a graphical user interface.

In some embodiments, the electronic stethoscope is configured for active noise cancelation.

In some embodiments, the electronic stethoscope includes a Hemholtz resonator configured to augment vibratory motion of sound waves generated by the leak.

Another implementation of the present disclosure is a method of detecting and repairing a negative pressure wound therapy (NPWT) dressing leak. The method includes, detecting the presence of one or more leaks in a NPWT dressing using a NPWT device. The NPWT device includes a first user interface, a negative pressure pump, a canister, and processing circuitry. The method includes, displaying, via the first user interface, instructions to locate the one or more leaks using an electronic stethoscope. The method includes determining one or more locations of the one or more leaks on the NPWT dressing using the electronic stethoscope, and displaying, via the first user interface, instructions for repairing the one or more leaks at the one or more locations determined using the electronic stethoscope.

In some embodiments, the instructions for repairing the one or more leaks include applying additional dressing to the one or more locations of the one or more leaks.

In some embodiments, the electronic stethoscope is configured to selectively filter frequency signals detected by a transducer of the electronic stethoscope.

Another implementation of the present disclosure is an advanced seal check system for negative pressure wound therapy (NPWT). The system comprises a dressing, a NPWT device, and an electronic stethoscope. The NPWT device includes a manifold layer for NPWT and a drape layer covering the manifold layer. The drape layer is configured to be sealingly coupled with skin surrounding the wound and defining a sealed inner volume of the dressing. The drape layer has an opening for drawing negative pressure at the sealed inner volume of the dressing. The NPWT device includes a negative pressure pump, a canister, a user interface, and a controller. The negative pressure pump is configured to generate the negative pressure at the sealed inner volume of the dressing. The canister is configured to collect fluid secreted by the wound during NPWT. The user interface includes a display. The controller includes processing circuitry configured to detect a leak in the dressing, determine a characteristic of the leak in the dressing, and display, via graphical user interface on the display, the characteristic of the leak in the dressing. The electronic stethoscope is configured to be communicably coupled with at least one of the NPWT device and a user device. The electronic stethoscope includes a transducer for detecting frequency signals generated by the leak in the dressing.

In some embodiments, the processing circuitry is further configured to display, via the graphical user interface on the display, instructions to locate the leak using the electronic stethoscope.

In some embodiments, the frequency signals detected by the transducer are displayed on the graphical user interface.

In some embodiments, the electronic stethoscope is configured to digitally filter the frequency signals detected by the transducer to isolate a second frequency signals emitted by the leak in the dressing.

Another implementation of the present disclosure is a method for checking a negative pressure wound therapy (NPWT) dressing. The method includes detecting a leak in a NPWT using a NPWT device, displaying, via a user interface, instructions for identify the leak using a listening device, and detecting a location of the leak in the NPWT dressing based on a sound detected by the listening device.

In some embodiments, the listening device comprises a sound transducing device configured to detect the sound, and the sound is associated with the leak.

In some embodiments, the listening device is associated with a user device.

In some embodiments, the listening device includes a Helmholtz resonator configured to augment vibratory motion of the sound.

In some embodiments, the user interface comprises a graphical user interface.

In some embodiments, the user interface is associated with a user device.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a block diagram of a wound therapy system including a therapy device coupled to a wound dressing via tubing, according to some embodiments.

FIG. 2 is a block diagram illustrating the therapy device of FIG. 1 in greater detail when the therapy device operates to draw a vacuum within a negative pressure circuit, according to some embodiments.

FIG. 3A is a block diagram illustrating the therapy device of FIG. 1 in greater detail when the therapy device operates to vent the negative pressure circuit, according to some embodiments.

FIG. 3B is a block diagram illustrating the therapy device of FIG. 1 in greater detail when the therapy device uses an orifice to vent the negative pressure circuit, according to some embodiments.

FIG. 4 is a block diagram illustrating the therapy device of FIG. 1 in greater detail when the therapy device operates to deliver instillation fluid to the wound dressing and/or a wound, according to some embodiments.

FIG. 5 is a perspective view of the therapy device of FIG. 1 paired with two electronic stethoscopes, according to some embodiments.

FIG. 6 is a block diagram illustrating the electronic stethoscope of FIG. 5 in greater detail, according to some embodiments.

FIG. 7 is a user interface of the therapy device of FIG. 1, according to some embodiments.

FIG. 8 is a user interface of the therapy device of FIG. 1, according to some embodiments.

FIG. 9 is a user interface of the therapy device of FIG. 1, according to some embodiments.

FIG. 10 is a user interface of the therapy device of FIG. 1, according to some embodiments.

FIG. 11 is a user interface of the therapy device of FIG. 1, according to some embodiments.

FIG. 12 is a user interface of the therapy device of FIG. 1, according to some embodiments.

FIG. 13 is a user interface of the therapy device of FIG. 1, according to some embodiments.

FIG. 14 is a sectional view of a wound dressing, according to some embodiments.

FIG. 15 is a perspective view of the therapy device of FIG. 1 coupled to a wound dressing via tubing, and communicably coupled to the electronic stethoscope of FIG. 6 and a user device, according to some embodiments.

FIG. 16 is a perspective view of the wound dressing of FIG. 14 and the electronic stethoscope of FIG. 5 paired to a user device having a user interface, according to some embodiments.

FIG. 17 is a perspective view of the wound dressing of FIG. 14 and the electronic stethoscope of FIG. 5 paired to a user device with an example user interface, according to some embodiments.

FIG. 18 is a perspective view of the wound dressing of FIG. 14 being repaired using instructions displayed on a user interface on the user device, according to some embodiments.

FIG. 19 is a perspective view of the wound dressing of FIG. 14 being checked using the electronic stethoscope of FIG. 5 and a user interface on a user device, according to some embodiments.

FIG. 20 is a flow diagram of detecting and repairing a leak in a wound dressing, according to some embodiments.

FIG. 21 is a flow diagram of detecting a leak in a wound dressing, according to some embodiments.

FIG. 22 is a block diagram of a therapy device configured to operate a pneumatic pump and an instillation pump to provide negative pressure wound therapy (NPWT) and an advanced seal check, according to some embodiments.

FIG. 23 is a perspective view of the user device of FIG. 5 configured to detect a leak, according to some embodiments.

### DETAILED DESCRIPTION

### Overview

Referring generally to the FIGURES, systems and methods for performing a seal check of a wound dressing utilized during negative pressure wound therapy (NPWT) are shown, according to various embodiments. NPWT can be provided to both facilitate healing progression of the wound, remove wound exudate, etc., and also to adjust, actuate, change, etc., a surface topology of the wound bed. To enable NPWT to provide effective treatment to a wound it is beneficial that a pneumatic seal is achieved. Leaks in the NPWT dressing and NPWT equipment can put a strain NPWT equipment, irritate a patient undergoing NPWT (e.g., through excessive pump noise, leak alarms, wound drying, etc.), and cause discomfort to the patient as caregivers work to find and seal the leak using traditional wound dressing check and repair methods (e.g., replacement of the wound dressing, pressing on random portions of the wound dressing to determine the location of the leak, etc.). Advantageously, the systems and methods described herein provide a less invasive, more accurate, and more efficient method of detecting and repairing a leak in a wound dressing utilized during NPWT.

### Wound Therapy System

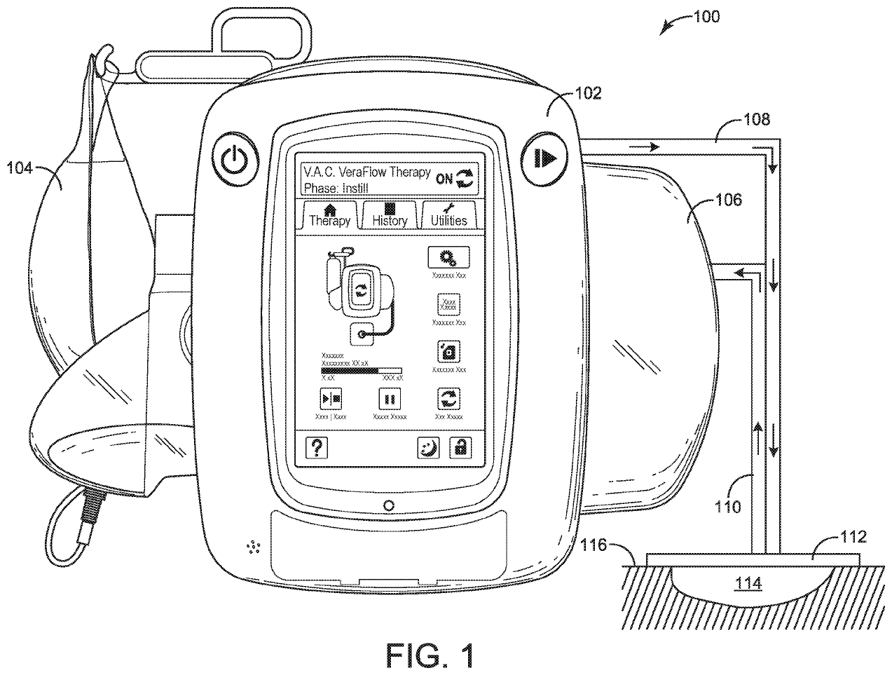

Referring now to FIGS. 1-4, a NPWT system 100 is shown, according to an exemplary embodiment. NPWT system 100 is shown to include a therapy device 102 fluidly connected to a wound dressing 112 via tubing 108 and 110. Wound dressing 112 may be adhered or sealed to a patient's skin (e.g., periwound tissue 116) surrounding a wound 114. Several examples of wound dressings 112 which can be used in combination with NPWT system 100 are described in detail in U.S. Pat. No. 7,651,484 granted Jan. 26, 2010, U.S. Pat. No. 8,394,081 granted Mar. 12, 2013, and U.S. Pat. No. 10,232,155 granted Mar. 19, 2019. The entire disclosure of each of these patents is incorporated by reference herein.

Therapy device 102 can be configured to provide negative pressure wound therapy by reducing the pressure at wound 114. Therapy device 102 can draw a vacuum at wound 114 (relative to atmospheric pressure) by removing wound exudate, air, and other fluids from wound 114. Wound exudate may include fluid that filters from a patient's circulatory system into lesions or areas of inflammation. For example, wound exudate may include water and dissolved solutes such as blood, plasma proteins, white blood cells, platelets, and red blood cells. Other fluids removed from wound 114 may include instillation fluid 105 previously delivered to wound 114. Instillation fluid 105 can include, for example, a cleansing fluid, a prescribed fluid, a medicated fluid, an antibiotic fluid, or any other type of fluid which can be delivered to wound 114 during wound treatment. Instillation fluid 105 may be held in an instillation fluid canister 104 and controllably dispensed to wound 114 via instillation fluid tubing 108. In some embodiments, instillation fluid canister 104 is detachable from therapy device 102 to allow canister 106 to be refilled and replaced as needed.

The fluids 107 removed from wound 114 pass through removed fluid tubing 110 and are collected in removed fluid canister 106. Removed fluid canister 106 may be a component of therapy device 102 configured to collect wound exudate and other fluids 107 removed from wound 114. In some embodiments, removed fluid canister 106 is detachable from therapy device 102 to allow canister 106 to be emptied and replaced as needed. A lower portion of canister 106 may be filled with wound exudate and other fluids 107 removed from wound 114, whereas an upper portion of canister 106 may be filled with air. Therapy device 102 can be configured to draw a vacuum within canister 106 by pumping air out of canister 106. The reduced pressure within canister 106 can be translated to wound dressing 112 and wound 114 via tubing 110 such that wound dressing 112 and wound 114 are maintained at the same pressure as canister 106.

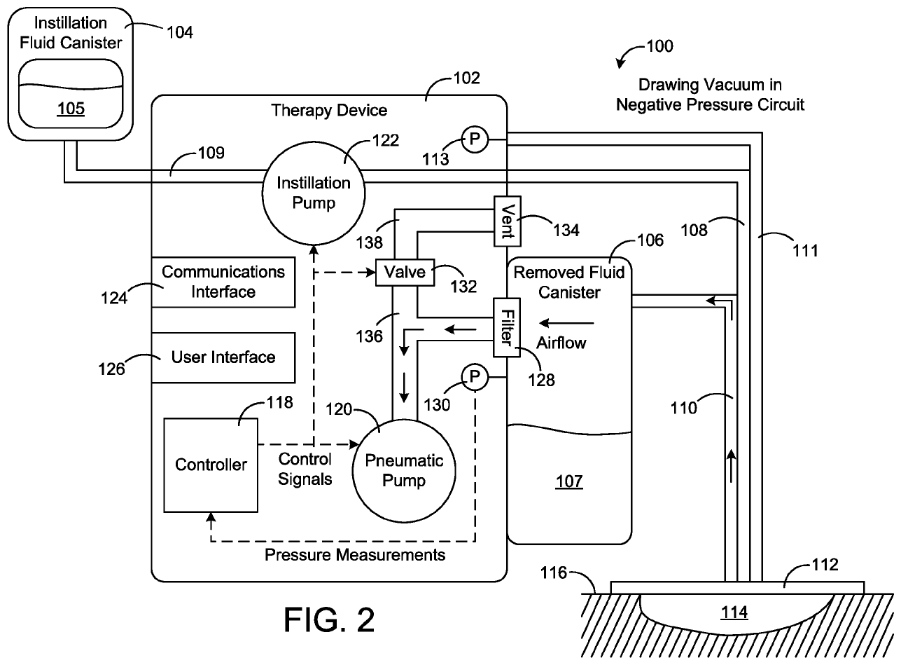

Referring particularly to FIGS. 2-4, block diagrams illustrating therapy device 102 in greater detail are shown, according to an exemplary embodiment. Therapy device 102 is shown to include a pneumatic pump 120, an instillation pump 122, a valve 132, a filter 128, and a controller 118. Pneumatic pump 120 can be fluidly coupled to removed fluid canister 106 (e.g., via conduit 136) and can be configured to draw a vacuum within canister 106 by pumping air out of canister 106. In some embodiments, pneumatic pump 120 is configured to operate in both a forward direction and a reverse direction. For example, pneumatic pump 120 can operate in the forward direction to pump air out of canister 106 and decrease the pressure within canister 106. Pneumatic pump 120 can operate in the reverse direction to pump air into canister 106 and increase the pressure within canister 106. Pneumatic pump 120 can be controlled by controller 118, described in greater detail below.

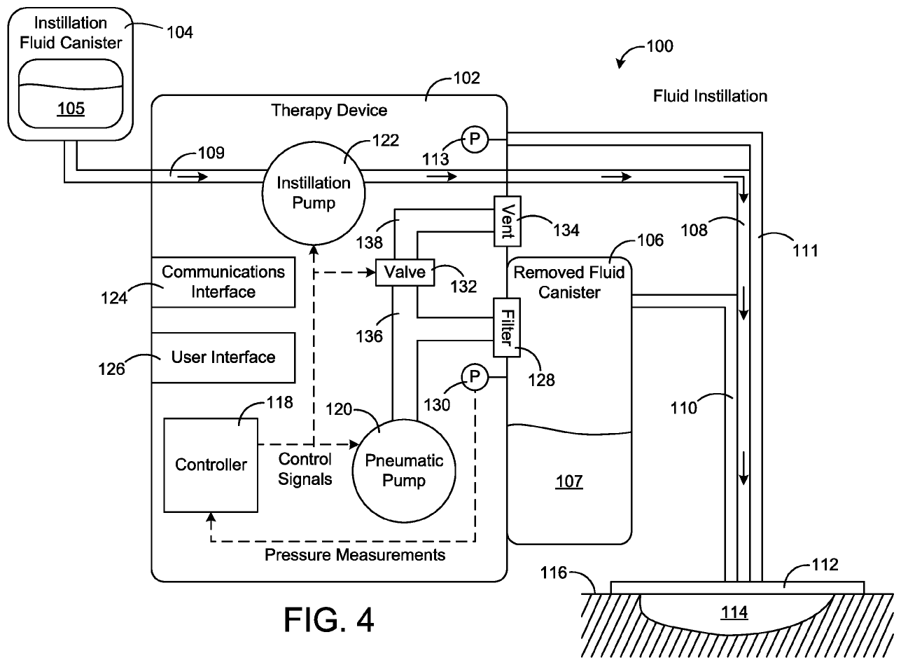

Similarly, instillation pump 122 can be fluidly coupled to instillation fluid canister 104 via tubing 109 and fluidly coupled to wound dressing 112 via tubing 108. Instillation pump 122 can be operated to deliver instillation fluid 105 to wound dressing 112 and wound 114 by pumping instillation fluid 105 through tubing 109 and tubing 108, as shown in FIG. 4. Instillation pump 122 can be controlled by controller 118, described in greater detail below.

Filter 128 can be positioned between removed fluid canister 106 and pneumatic pump 120 (e.g., along conduit 136) such that the air pumped out of canister 106 passes through filter 128. Filter 128 can be configured to prevent liquid or solid particles from entering conduit 136 and reaching pneumatic pump 120. Filter 128 may include, for example, a bacterial filter that is hydrophobic and/or lipophilic such that aqueous and/or oily liquids will bead on the surface of filter 128. Pneumatic pump 120 can be configured to provide sufficient airflow through filter 128 that the pressure drop across filter 128 is not substantial (e.g., such that the pressure drop will not substantially interfere with the application of negative pressure to wound 114 from therapy device 102).

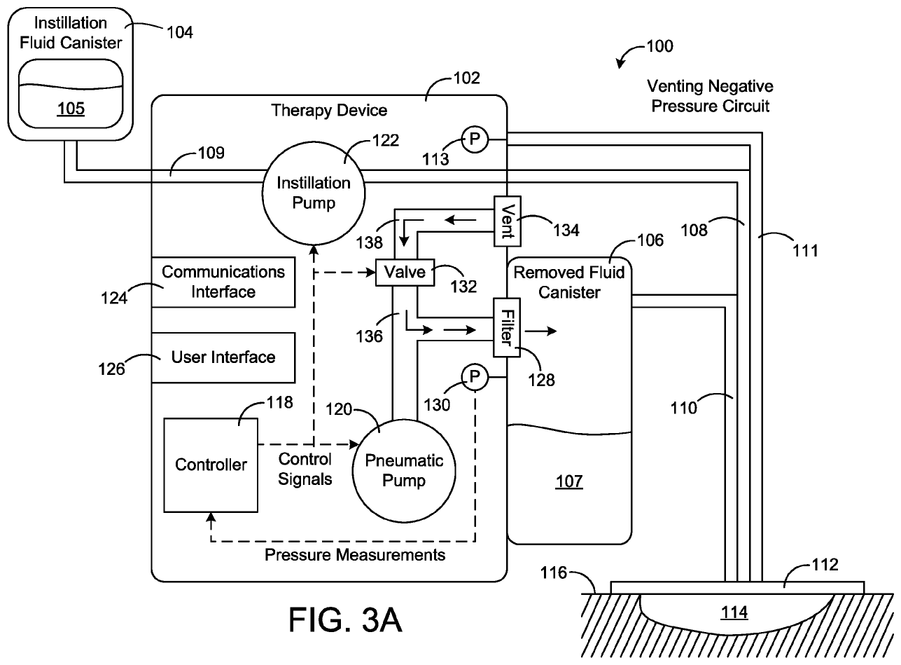

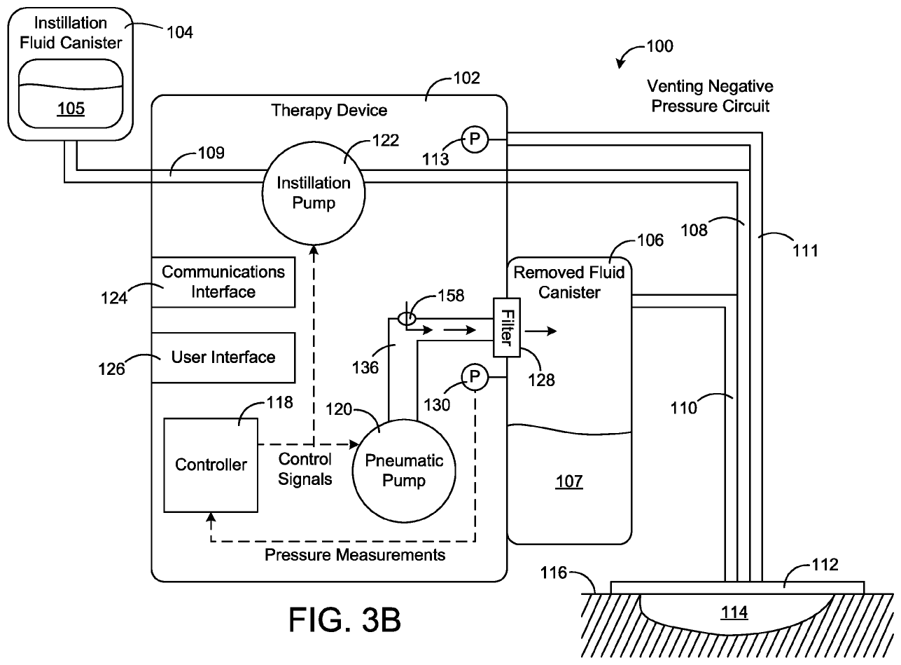

In some embodiments, therapy device 102 operates a valve 132 to controllably vent the negative pressure circuit, as shown in FIG. 3A. Valve 132 can be fluidly connected with pneumatic pump 120 and filter 128 via conduit 136. In some embodiments, valve 132 is configured to control airflow between conduit 136 and the environment around therapy device 102. For example, valve 132 can be opened to allow airflow into conduit 136 via vent 134 and conduit 138, and closed to prevent airflow into conduit 136 via vent 134 and conduit 138. Valve 132 can be opened and closed by controller 118, described in greater detail below. When valve 132 is closed, pneumatic pump 120 can draw a vacuum within a negative pressure circuit by causing airflow through filter 128 in a first direction, as shown in FIG. 2. The negative pressure circuit may include any component of NPWT system 100 that can be maintained at a negative pressure when performing negative pressure wound therapy (e.g., conduit 136, removed fluid canister 106, tubing 110, wound dressing 112, and/or wound 114). For example, the negative pressure circuit may include conduit 136, removed fluid canister 106, tubing 110, wound dressing 112, and/or wound 114. When valve 132 is open, airflow from the environment around therapy device 102 may enter conduit 136 via vent 134 and conduit 138 and fill the vacuum within the negative pressure circuit. The airflow from conduit 136 into canister 106 and other volumes within the negative pressure circuit may pass through filter 128 in a second direction, opposite the first direction, as shown in FIG. 3A.

In some embodiments, therapy device 102 vents the negative pressure circuit via an orifice 158, as shown in FIG. 3B. Orifice 158 may be a small opening in conduit 136 or any other component of the negative pressure circuit (e.g., removed fluid canister 106, tubing 110, tubing 111, wound dressing 112, etc.) and may allow air to leak into the negative pressure circuit at a known rate. In some embodiments, therapy device 102 vents the negative pressure circuit via orifice 158 rather than operating valve 132. Valve 132 can be omitted from therapy device 102 for any embodiment in which orifice 158 is included. The rate at which air leaks into the negative pressure circuit via orifice 158 may be substantially constant or may vary as a function of the negative pressure, depending on the geometry of orifice 158. For embodiments in which the leak rate via orifice 158 is variable, controller 118 can use a stored relationship between negative pressure and leak rate to calculate the leak rate via orifice 158 based measurements of the negative pressure. Regardless of whether the leak rate via orifice 158 is substantially constant or variable, the leakage of air into the negative pressure circuit via orifice 158 can be used to generate a pressure decay curve for use in estimating the volume of wound 114.

In some embodiments, the controller 118 is configured to detect and determine a leakage from the wound dressing 112 in combination with orifice 158. For example, if the leak rate of orifice 158 is known and controlled, the controller 118 may trigger an alarm or otherwise notify a user when a determined or measured leak rate is higher than the known leak rate of the orifice 158. In some embodiments, the controller 118 may store an expected negative pressure loss rate associated with typical NPWT. For example, if a negative pressure is drawn to 125 mm Hg, and the negative pressure decreases to 100 mm Hg over period of time, the controller 118 may determine if the rate is expected (e.g., predetermined, typical, controlled, etc.), or abnormal (e.g., unexpected, undesirable, etc.). In some embodiments, an expected loss in negative pressure is due an expected amount of fluid (e.g., wound exudate) being drawn from the wound 114.

In some embodiments, therapy device 102 includes a variety of sensors. For example, therapy device 102 is shown to include a pressure sensor 130 configured to measure the pressure within canister 106 and/or the pressure at wound dressing 112 or wound 114. In some embodiments, therapy device 102 includes a pressure sensor 113 configured to measure the pressure within tubing 111. Tubing 111 may be connected to wound dressing 112 and may be dedicated to measuring the pressure at wound dressing 112 or wound 114 without having a secondary function such as channeling installation fluid 105 or wound exudate. In various embodiments, tubing 108, 110, and 111 may be physically separate tubes or separate lumens within a single tube that connects therapy device 102 to wound dressing 112. Accordingly, tubing 110 may be described as a negative pressure lumen that functions apply negative pressure wound dressing 112 or wound 114, whereas tubing 111 may be described as a sensing lumen configured to sense the pressure at wound dressing 112 or wound 114. Pressure sensors 130 and 113 can be located within therapy device 102, positioned at any location along tubing 108, 110, and 111, or located at wound dressing 112 in various embodiments. Pressure measurements recorded by pressure sensors 130 and/or 113 can be communicated to controller 118. Controller 118 use the pressure measurements as inputs to various pressure testing operations and control operations performed by controller 118.

Controller 118 can be configured to operate pneumatic pump 120, instillation pump 122, valve 132, and/or other controllable components of therapy device 102. In some embodiments, controller 118 performs a pressure testing procedure by applying a pressure stimulus to the negative pressure circuit. For example, controller 118 may instruct valve 132 to close and operate pneumatic pump 120 to establish negative pressure within the negative pressure circuit. Once the negative pressure has been established, controller 118 may deactivate pneumatic pump 120. Controller 118 may cause valve 132 to open for a predetermined amount of time and then close after the predetermined amount of time has elapsed.

In some embodiments, therapy device 102 includes a user interface 126. User interface 126 may include one or more buttons, dials, sliders, keys, user interactive displays, user interactive surfaces, or other input devices configured to receive input from a user. User interface 126 may also include one or more displays (e.g., liquid-crystal display, light emitting diode display, organic light emitting diode display, electrophoretic display, etc.), speakers, light emitting devices, and/or other output devices configured to provide information to a user.

In some embodiments, therapy device 102 includes a data communications interface 124 (e.g., a USB port, a wireless transceiver, etc.) configured to receive and transmit data. The communications interface 124 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, communications interface 124 can include a USB port or an Ethernet card and port for sending and receiving data via an Ethernet-based communications link or network. In another example, communications interface 124 can include a Wi-Fi transceiver for communicating via a wireless communications network or cellular or mobile phone communications transceivers. In another example, communications interface 124 can include a short-range wireless transmitter (e.g., a Bluetooth wireless transmitter, etc.) for communicating via a short range wireless communication transceivers.

### Electronic Stethoscope

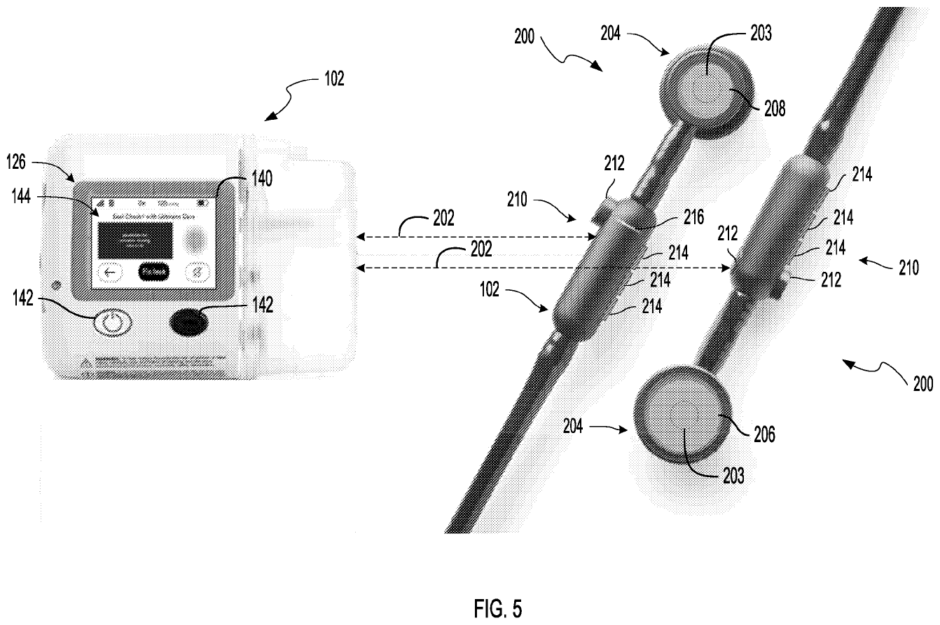

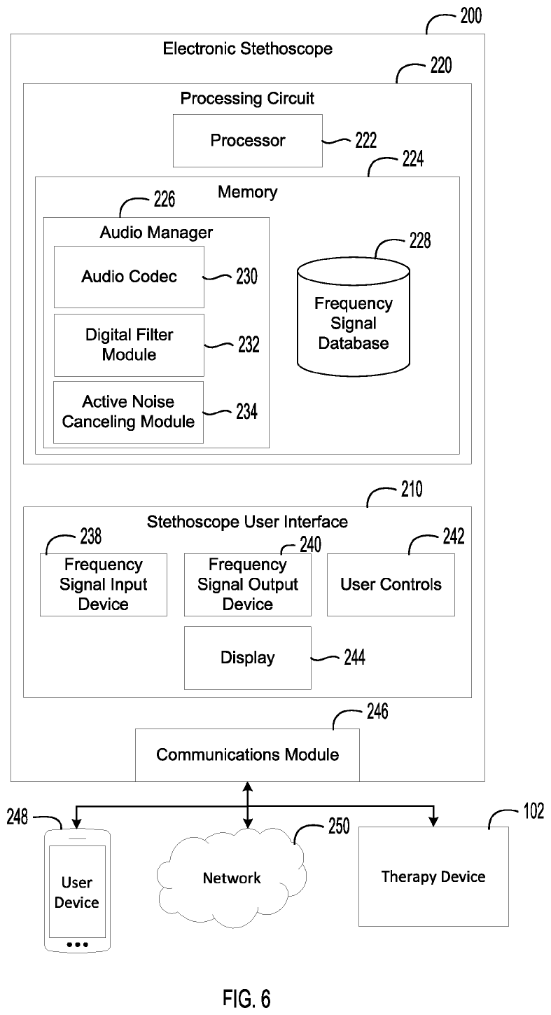

Referring to FIGS. 5 and 6, a listening device (e.g., a sound detecting device, a sound transducing device, a frequency signal transducing device, a microphone equipped device, etc.), shown as electronic stethoscope 200 is shown, according to some embodiments. In some embodiments, any of the embodiments of the electronic stethoscope 200 and therapy device 102 described herein are useable with the NPWT system 100 as described in greater detail above with reference to FIGS. 1-4. In some embodiments, the electronic stethoscope 200 and the therapy device 102 as described herein are provided as a system for providing NPWT to a wound.

Referring particularly to FIG. 5, a therapy device 102 and two electronic stethoscopes 200 are shown, according to some embodiments. As shown, electronic stethoscopes 200 are communicably coupled to therapy device 102 by connection 202. In some embodiments, connection 202 a wired or wireless connection. In some embodiments, connection 202 is a direct connection (e.g., short-range wireless connection, short-range wireless radio connection, hardwire connection, etc.) between therapy device 102 and electronic stethoscopes 200. In some embodiments, connection 202 is an indirect connection and data is exchanged over a network. For example, connection 202 may include communications over a local area network (LAN), wireless local area network (WLAN), wide area network (WAN), cellular network, and other suitable networks for communicating data between at least electronic stethoscopes 200 and therapy device 102.

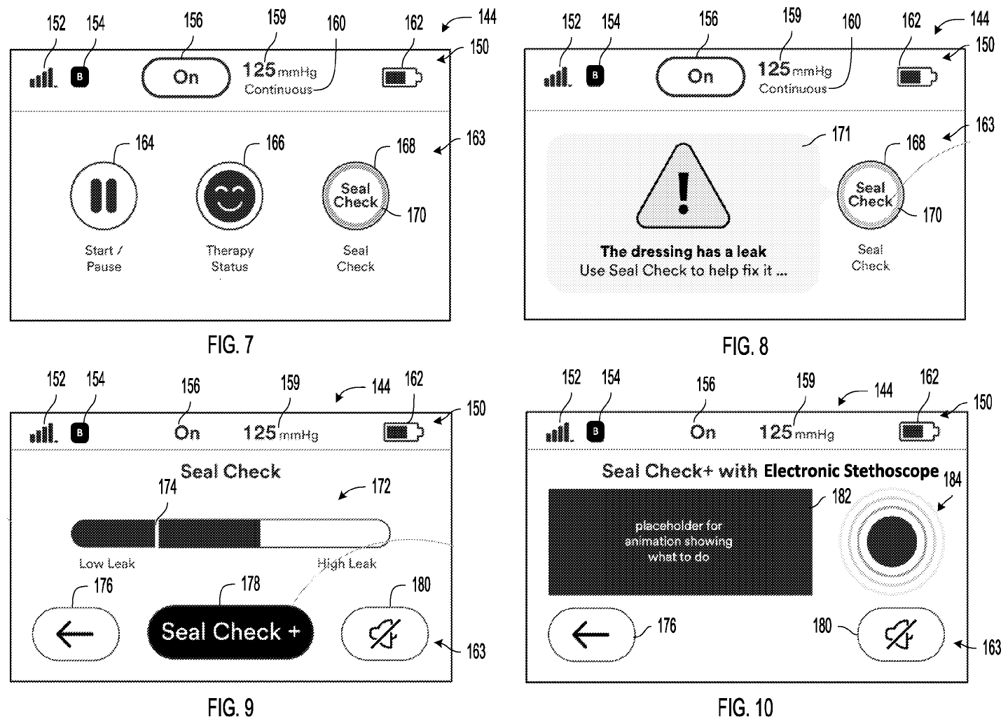

As shown, the therapy device 102 includes a user interface 126. In some embodiments, the user interface 126 includes a display 140 and buttons 142. In some embodiments, the display 140 is a touch sensitive display (e.g., touchscreen display, etc.). As shown, the display 140 is displaying a graphical user interface 144. The graphical user interface 144 is described in greater detail with respect to FIGS. 7-13 below.

In some embodiments, the electronic stethoscope 200 includes a frequency signal transducer 203 (e.g., sound transducer, microphone, etc.) housed by a transducer enclosure (e.g., chest piece, resonator, etc.), shown as bell 204. In some embodiments, bell 204 includes a Helmholtz resonator (e.g., Helmholtz oscillator), configured to augment the amplitude of sound produced by a pneumatic leak in wound dressing 112 for a range of frequencies. In some embodiments, the augmented amplitude of sound (e.g., frequency signals, soundwaves, etc.) produced by the pneumatic leak in the wound dressing 112 facilitates the frequency signal transducer 203 transducing the sound. For example, a Helmholtz resonator may have a resonant frequency at one or more specific frequencies between 20 Hz to 40 kHz to enhance detection of sound within the range. In some embodiments, a Helmholtz resonator may be selected having a resonant frequency between 20 kHz and 40 kHz. In some embodiments, frequency signal transducer 203 is configured to detect leaks which produce ultrasound sound waves (e.g., sound waves having a frequency between approximately 20 kHz to approximately 100 kHz). In some embodiments, bell 204 includes a first diaphragm 206 (e.g., an adult diaphragm, an adult resonator, etc.) and a second diaphragm 208 (e.g., a child diaphragm, a child resonator, etc.). In some embodiments, first diaphragm 206 is larger than second diaphragm 208. In some embodiments, first diaphragm 206 is more sensitive to nearby vibrations than second diaphragm 208. In some embodiments, electronic stethoscope 200 does not include a diaphragm.

In some embodiments, electronic stethoscope 200 is configured to support an analogue mode (e.g., mechanical stethoscope mode, etc.) and an amplified listening mode (e.g., a filtered signal mode, a selectively amplified listening mode, an electronically enhanced listening mode, an electronically recorded and electronically reproduced listening mode, etc.). In such embodiments, electronic stethoscope 200 may be similar to a traditional stethoscope having a chest piece (e.g., bell 204), tubing, and a headset including ear tubes and ear tips, which may be capable of functioning without electrical power. In some embodiments, electronic stethoscope 200 includes electronic features such as a frequency signal transducer, a signal amplifier, a processing circuit, a memory, a communications interface, a filter, and a user interface. In some embodiments, electronic stethoscope 200 does not resemble a traditional stethoscope. For example, in some embodiments, electronic stethoscope 200 may not include tubing or a headset. In another example, electronic stethoscope 200 may include only the bell 204 configured to be communicably coupled to external devices (e.g., therapy device 102).

As shown in FIG. 5, electronic stethoscope 200 includes a stethoscope user interface 210. In some embodiments, stethoscope user interface 210 includes a toggle switch 212. In some embodiments, the toggle switch 212 is configured to transition the electronic stethoscope 200 between an analogue mode and an electronically enhanced listening mode. In some embodiments, the amplified listening mode is at least one of a filtered signal mode (e.g., a digitally filtered listening mode), a selectively amplified listening mode, and an electronically recorded and electronically reproduced listening mode. In some embodiments, stethoscope user interface 210 includes buttons 214. In some embodiments, buttons 214 include an up button (e.g., a plus button, an advance button, an increase button, etc.), a down button (e.g., a minus button, a back button, a decrease button, etc.), and a multifunctional button to allow a user to interact with the electronic stethoscope 200. For example, a user may increase or decrease the volume of sound output by the electronic stethoscope 200 by interacting with buttons 214. In some embodiments, a user may pair the electronic stethoscope 200 with a therapy device 102 or a user device by interacting with buttons 214. In some embodiments, the electronic stethoscope 200 includes an indicator 216. In some embodiments, the indicator 216 includes a multicolored light emitting diode (LED). In some embodiments, the indicator 216 is configured to emit a number of colors (e.g., blue, orange, yellow, green, etc.) and light patterns (e.g., light flashes, light intensity patterns, etc.) to communicate a status of the electronic stethoscope 200 to the user.

As shown in FIG. 6, electronic stethoscope 200 includes a processing circuit 220, according to some embodiments. In some embodiments, the processing circuit 220 includes a processor 222 and a memory 224 (e.g., memory, memory unit, storage device, etc.). In some embodiments, the processor 222 is implemented as a general purpose processor, an application specific integrated circuit (ASIC), one or more field programmable gate arrays (FPGAs), a group of processing components, or other suitable electronic processing components.

In some embodiments, the memory 224 includes one or more devices (e.g., RAM, ROM, Flash memory, hard disk storage, etc.) for storing data and/or computer code for completing or facilitating the various processes described in the present application. The memory 224 can be or include volatile memory or non-volatile memory. The memory 224 can include database components, object code components, script components, or any other type of information structure for supporting the various activities and information structures described in the present application. According to some embodiments, the memory 224 is communicably connected to processor 222 via the processing circuit 220 and includes computer code for executing (e.g., by processing circuit 220 and/or processor 222) one or more processes described herein. In some embodiments, the memory 224 may be a local or remote memory.

Still referring to FIG. 6, the memory 224 includes an audio manager 226, according to some embodiments. In some embodiments, the audio manager 226 includes modules for encoding and processing frequency signals detected by the frequency signal transducer 203. In some embodiments, audio manager 226 includes an audio codec 230 that is capable of encoding and decoding audio signals. In some embodiments, the audio codec 230 is utilized to reduce the required bandwidth for transmission of the frequency signals detected by the electronic stethoscope 200. In some embodiments, the audio manager 226 may send audio signals in an encoded format to the frequency signal database 228, and may decode audio signals stored in the frequency signal database 228 using the audio codec 230 (e.g., to support audio playback).

In some embodiments, the audio manager 226 includes a digital filter module 232. In some embodiments, the digital filter module 232 is an electronic filter (e.g., low-pass filter, high-pass filter, etc.) that is configured to pass a range of frequencies and attenuate other frequencies (e.g., frequencies outside of the range of pass frequencies). In some embodiments, the digital filter module 232 is configured to pass a specific range of frequencies associated with a leak in the wound dressing 112.

In some embodiments, audio manager 226 includes an active noise canceling module 234. In some embodiments, active noise canceling module 234 is configured to reduce or remove unwanted frequency signals (e.g., background noise, pump noise, etc.) by generating a destructive signal (e.g., a frequency signal 180° out of phase of the unwanted frequency signals) which reduces or cancels the unwanted frequency signals. In some embodiments, the active noise canceling module 234 is activated to provide a clearer listening experience for the user. In some embodiments, the active noise canceling module 234 temporarily or permanently stores the frequency signals transduced by the frequency signal transducer 203 in frequency signal database 228.

Still referring to FIG. 6, the electronic stethoscope 200 includes a stethoscope user interface 210. As shown, the stethoscope user interface 210 includes a frequency signal input device (e.g., frequency signal transducer, microphone, frequency signal transducer 203, etc.) and a frequency signal output device 240. In some embodiments, the frequency signal input device 238 is housed in a bell 204 and is configured to detect vibrations (e.g., soundwaves) nearby the frequency signal input device 238. In some embodiments, frequency signal input device 238 is configured to send frequency signals to audio manager 226 and frequency signal database 228. In some embodiments, the frequency signal input device 238 includes the frequency signal transducer 203. In some embodiments, the frequency signal output device 240 is a speaker, headphones, earplugs, display, one or more LEDs, or other device capable of reproducing or representing frequency signals. In some embodiments, the frequency signal output device is configured to output frequency signals that have been processed through digital filter module 232 and active noise canceling module 234. In some embodiments, the frequency signal output device 240 is configured to communicate the frequency signals detected by the frequency signal transducer 203 using the communications module 246.

In some embodiments, stethoscope user interface 210 includes user controls 242 such as toggle switch 212, buttons 214, dials, keypad, switches, touch sensitive surfaces, etc. In some embodiments, stethoscope user interface 210 includes a display 244. The display 244 may be at least one of an liquid-crystal display (LCD) display, organic light emitting diode (OLED) display, light emitting diode (LED) display, or other display capable of presenting information to the user. In some embodiments, display 244 is a user interactive display (e.g., touchscreen display) which presents a graphical user interface and receives user inputs via a user interactive surface (e.g., touch sensitive surface) and user controls 242 to thereby enable a user to easily and intuitively interact with electronic stethoscope 200. In some embodiments, display 244 is an indicator 216.

In some embodiments, the electronic stethoscope 200 includes a communications module 246. In some embodiments, the communications module 246 is the same as or similar to the communications interface 124. The communications module 246 may comprise software and hardware for communicably connecting the electronic stethoscope 200 to a user device 248, a network 250 and/or the therapy device 102, according to some embodiments.

Still referring to FIG. 6, the user device 248 is any electronic device that allows a user to view information from the electronic stethoscope 200 and/or interact with the electronic stethoscope 200, according to some embodiments. Examples of user devices include, but are not limited to, mobile phones, electronic tablets, laptops, desktop computers, workstations, smart watches, and other types of electronic devices. Accordingly, user device 248 may include a user input device, such as a keyboard, a touchscreen display, a keypad, buttons, switches, etc. The user device 248 may present a graphical user interface on an on-board display, which may supplement display 244. For example, in some embodiments, electronic stethoscope 200 does not include display 244 and instead uses a display on a user device 248 to present information. In some embodiments, stethoscope user interface 210 includes only a frequency signal input device 238 and user controls 242, and frequency signal output device 240 and display 244 are located on the user device 248.

In some embodiments, the user device 248 includes a communication module capable of connecting to multiple user devices and user accessories (e.g., wireless headphones, smart watches, etc.). In such embodiments, user device 248 may communicate information from electronic stethoscope 200 to the connected user devices and user accessories, and may also receive user inputs from the connected user devices and user accessories. For example, a smart watch may be connected to a user device 248 and a user may interact with the smart watch to retrieve and view data and provide inputs to the electronic stethoscope 200. In another example, wireless headphones (e.g., earbuds) may be paired to user device 248 which may function as the frequency signal output device 240. In some embodiments, a frequency signal output device 240 (e.g., wireless earbuds, wireless headphones, wireless speaker, etc.) may be communicably connected (e.g., paired) directly to the electronic stethoscope 200 through the communications module 246. In some embodiments, user device 248 includes a processing circuit having a processor and a memory. In some embodiments, the processing circuit on-board the user device 248 is the same as or similar to the processing circuit 220, processor 222, and memory 224.

In some embodiments, network 250 may include a local area network (LAN), wireless local area network (WLAN), wide area network (WAN), cellular network, and other suitable networks suitable for communicating data between at least the electronic stethoscope 200, the therapy device 102, and/or the user device 248. In some embodiments, network 250 is connected to a cloud-based server which may be configured to store frequency signals similar to or the same as frequency signal database 228. In some embodiments, the cloud based server is configured to collect data that facilitates software and hardware improvement.

### Seal Check Using an Electronic Stethoscope

Referring now to FIGS. 7-13, the graphical user interface 144 is shown, according to some embodiments. In some embodiments, the graphical user interface 144 includes a system status portion 150. In some embodiments, the system status portion 150 includes a communications signal strength indicator 152. In some embodiments, the communications signal strength indicator 152 is configured to indicate the strength of a signal received by the communications interface 124. In some embodiments, the system status portion 150 includes a communications type indicator 154 (e.g., Bluetooth, Wi-Fi, Cellular, etc.). In some embodiments, the system status portion 150 includes a therapy status indicator 156 which may be configured to indicate the current state of the therapy device 102 (e.g., on, off, standby, hold, etc.). In some embodiments, system status portion 150 includes a therapy pressure indicator 159 and a therapy setting indicator 160. In some embodiments, the therapy pressure indicator 159 is configured to indicate the current pressure setting of the therapy device (e.g., 125 mmHg, 100 mmHg, etc.). In some embodiments, the therapy setting indicator 160 is configured to indicate the current therapy type setting of the therapy device 102 (e.g., continuous therapy, periodic therapy, static pressure, dynamic pressure, oscillating pressure, stepped pressure, etc.). In some embodiments, therapy device 102 includes an internal power source, and the battery charge level is indicated by the battery charge level indicator 162.

Referring particularly to FIG. 7, the graphical user interface 144 includes user selectable options 163. In some embodiments, the user selectable options 163 include a therapy start/pause button 164. The therapy start/pause button 164 may switch between a play symbol and a pause symbol to indicate the state that will be activated upon a user interacting with start/pause button 164. For example, a pause symbol may be displayed while the therapy is operating (e.g., drawing negative pressure at the wound, etc.) to indicate that interacting with start/pause button 164 will pause the therapy (e.g., stop drawing negative pressure at the wound). In some embodiments, a user may interact with start/pause button 164 to start and stop the application of a negative pressure to the wound 114.

In some embodiments, user selectable options 163 include a therapy status button 166. In some embodiments, therapy status button 166 may indicate the system status using a universally symbolic system (e.g., a depiction of a smiling or frowning face). A user may interact with therapy status button 166 to access therapy status information (e.g., duration, history, pressure settings, treatment settings, etc.) and interfaces for adjusting therapy status information, according to some embodiments.

In some embodiments, the user selectable options 163 include a seal check button 168. The seal check button 168 may include a seal check indication ring 170 which may indicate the current status of the therapy system by displaying a color (e.g., green, yellow, orange, red, etc.). For example, seal check indication ring 170 may be green when therapy device 102 is not detecting an unexpected leak in the negative pressure circuit, and may be yellow when therapy device 102 is detecting an unexpected leak in the negative pressure circuit, according to some embodiments.

Referring particularly to FIG. 8, therapy device 102 is detecting an unexpected leak in the negative pressure circuit and seal check indication ring 170 is yellow. In some embodiments, an informational window may 171 may display to provide a user with additional information about the unexpected leak (e.g., likely causes, alarm duration, leak detection start time, etc.). In some embodiments, therapy device 102 is configured to generate an auditory alarm and/or other notification (e.g., a push notification on user device 248, an email, a text, etc.) in response to therapy device 102 detecting an unexpected leak in the negative pressure circuit. In some embodiments, informational window 171 may include information about potential leak locations and alarm causes (e.g., canister 106 removed, leak in wound dressing 112, poor connection between tubing, etc.).

Referring particularly to FIG. 9, a user has interacted with the seal check button 168 of FIG. 8, and a seal check leak bar 172 is displayed. In some embodiments, the seal check leak bar 172 may be configured to display the severity of an unexpected leak on a low to high scale. In some embodiments, the seal check leak bar 172 may display the unexpected leak rate (e.g., cc/min). As shown, the seal check leak bar 172 includes a leak threshold 174. In some embodiments, the leak threshold 174 may indicate a threshold of a leak rate that is acceptable for operation using an internal power source (e.g., battery) versus utility power (e.g., mains power). For example, a leak rate higher than leak threshold 174 may cause an unacceptable battery discharge rate which may lead to an incomplete NPWT cycle. In some embodiments, leak threshold 174 is a predetermined threshold that corresponds to an acceptable pump duty cycle (e.g., a pump duty cycle that does not cause irritation or discomfort to a patient).

In some embodiments, graphical user interface 144 includes a back button 176, a seal check+ button 178, and an alarm silence button 180. In some embodiments, back button 176 is configured to allow a user to return to a previously configured display. For example, a user may interact with back button 176 to return to graphical user interface 144 of FIG. 8. In some embodiments, alarm silence button 180 is configured to silence (e.g., stop) an auditory alarm generated by therapy device 102. In some embodiments, a user may utilize seal check leak bar 172 to locate and repair a leak using a guess and check method. For example, a user may cover and/or press on portions of wound dressing 112 while monitoring seal check leak bar 172 to determine if the portion of wound dressing 112 being covered/pressed is the location of one or more leaks. In some embodiments, seal check+ button 178 is configured to facilitate a user pairing the electronic stethoscope 200 to therapy device 102.

Referring particularly to FIG. 10, a user has interacted with the seal check+ button 178 and an instruction window 182 is displayed. Instruction window 182 may display user executable instructions (e.g., user steps) for performing an advanced seal check in at least a written or video format. For example, instruction window 182 may display an animation, list of steps, or a combination of an animation and list of steps for performing an advanced seal check using an electronic stethoscope. Particularly in FIG. 10, instructions for pairing an electronic stethoscope 200 to therapy device 102 are displayed in instruction window 182.

In some embodiments, graphical user interface 144 includes a stethoscope seal indicator 184. In some embodiments, the stethoscope seal indicator 184 may use colors to indicate if the electronic stethoscope is paired (e.g., communicably connected). For example, stethoscope seal indicator 184 may be black or gray before pairing, and may change colors after the device displaying the graphical user interface (e.g., user device 248, therapy device 102, etc.) is paired with the electronic stethoscope 200. In some embodiments, the appearance (e.g., size, shape, diameter, etc.) of seal indicator 184 may correspond to the leak rate. For example, if the leak rate is low, stethoscope seal indicator 184 may be small (e.g., have a small diameter), and if the leak rate is high, stethoscope seal indicator 184 may be large (e.g., have a large diameter).

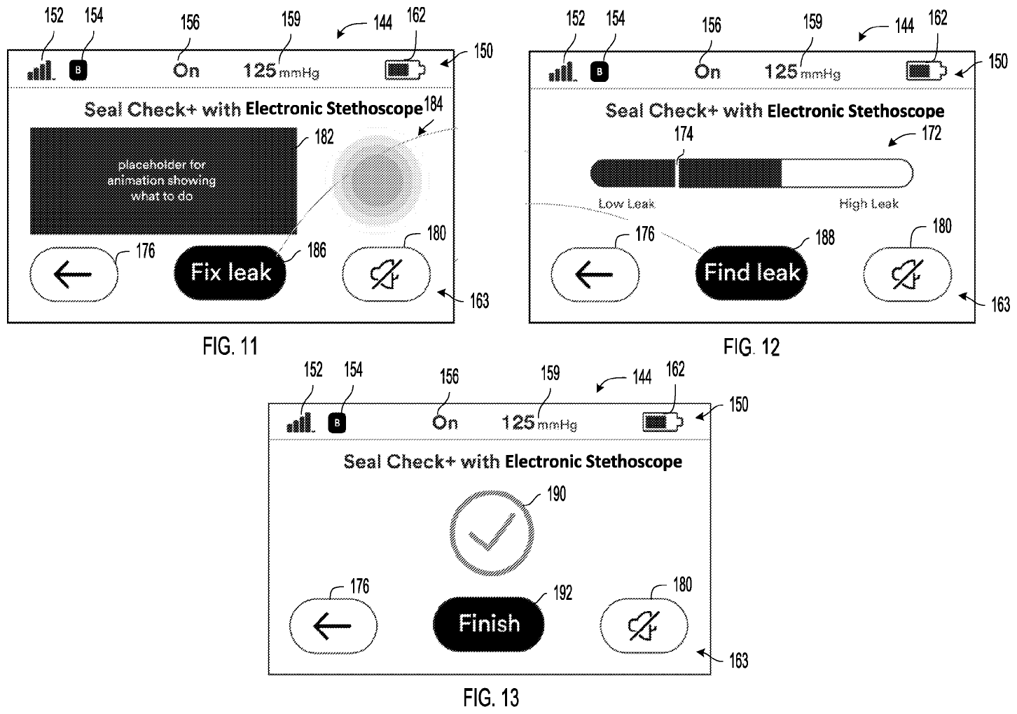

Referring particularly to FIG. 11, the electronic stethoscope 200 has been paired to the therapy device 102 and the paired status is indicated by stethoscope seal indicator 184 (e.g., seal indicator 184 is yellow). The instruction window 182 may display instructions for determining the location of the leak using the electronic stethoscope 200. For example, the instruction window 182 may display instructions for sweeping (e.g., scanning, moving, etc.) the frequency signal transducer 203 around the wound dressing 112. In some embodiments, the bell 204 does not contact the wound dressing 112 while searching for a leak in the wound dressing 112, which may advantageously facilitate a more comfortable seal check process. In some embodiments, graphical user interface 144 includes a fix leak button 186. In some embodiments, the fix leak button 186 may be visible when a leak has been detected by the electronic stethoscope 200. For example, after electronic stethoscope 200 detects a leak (e.g., a frequency signal determined to be associated with a leak) fix leak button 186 may be visible and selectable by a user. A user may interact with the fix leak button 186 and the instruction window 182 may display steps executable by a user to fix (e.g., repair) the leak, as described in greater detail below.

Referring particularly to FIG. 12, a user has located the leak using the electronic stethoscope 200 and has interacted with the fix leak button 186, according to some embodiments. The seal check leak bar 172 may be displayed on the graphical user interface 144 to facilitate a user monitoring the efficacy of the repair applied to the wound. For example, a user may be instructed to apply additional dressing (e.g., drape, foam, etc.) to the wound dressing 112 at the location where the leak was detected by the electronic stethoscope 200 and seal check leak bar 172 may display a change in a characteristic of the leak (e.g., the leak rate) after the repair has been applied. In some embodiments, find leak button 188 may be displayed to facilitate a user returning to graphical user interface 144 as shown in FIG. 11. For example, a user may reduce the leak rate by applying a first repair, but the leak rate may still be above the leak threshold 174, and a user may apply a second (e.g., third, fourth, fifth, etc.) repair to further reduce the leak rate at the same or different locations on the wound dressing 112. In some embodiments, the leak rate may still be above leak threshold 174 and no leaks may be detected around the wound dressing 112. In such embodiments, the user may be instructed (e.g., by instruction window 182) to check for leaks in other portions of the negative pressure circuit (e.g., tubing 110, canister 106, etc.) using the electronic stethoscope 200.

Referring particularly to FIG. 13, a user has reduced the leak rate below the leak threshold 174 by applying a repair to the wound dressing 112 and the graphical user interface 144 is presenting a success symbol 190 (e.g., a green check mark) indicating a successful repair has been made. In some embodiments, a user may interact with finish button 192 to exit the seal check user interface and return to the graphical user interface 144 shown in FIG. 7.

### NPWT Wound Dressing

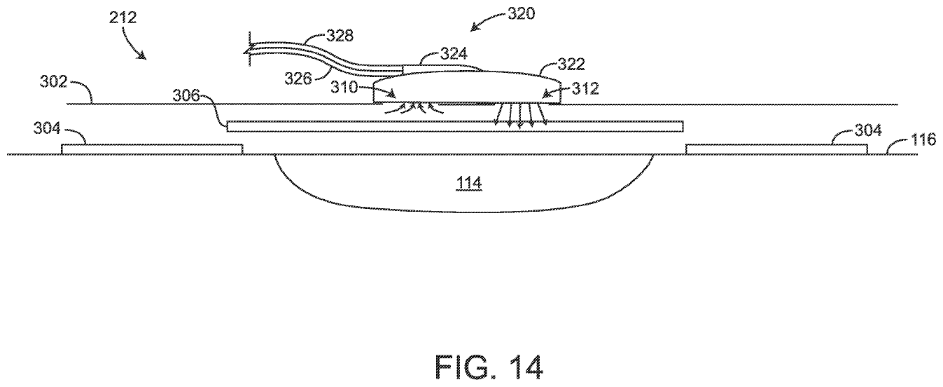

Referring now to FIG. 14, a wound dressing 112 is shown according to some embodiments. In some embodiments, the wound dressing 112 as shown in FIGS. 14-19 is similar to or the same as the wound dressing 112 described in greater detail above with reference to FIGS. 1-4. In some embodiments, the wound dressing 112 is configured to facilitate NPWT for a wound 114 that the wound dressing 112 covers. In some embodiments, the wound dressing 112 includes a drape 302, a skin interface layer 304, and a manifold layer 306, according to some embodiments. In some embodiments, the skin interface layer 304 is configured to directly contact, abut, engage, etc. an exterior surface of the patients skin surround the wound 114 (e.g., periwound tissue 116). In some embodiments, the skin interface layer 304 is a Dermatac™ material that is manufactured by 3M™. In some embodiments, the skin interface layer 304 is configured to sealingly couple on one side with the periwound tissue 116, and on an opposite side with the drape 302. In some embodiments, the drape 302 and the skin interface layer 304 are configured to cooperatively define an inner volume that includes the wound 114 therewithin. In some embodiments, the skin interface layer 304 and the drape 302 are integrally formed and provided as a unitary member. In some embodiments, the skin interface layer 304 and the drape 302 are Dermatac™ Drape with V.A.C.® Granufoam™ as manufactured by 3M™.

In some embodiments, manifold layer 306 is a foam layer that is configured to facilitate distribution of negative pressure throughout the inner volume defined by the drape 302 and the skin interface layer 304. In some embodiments, the manifold layer 306 is positioned within the inner volume (e.g., a sealed inner volume). In some embodiments, the manifold layer 306 is positioned directly below the drape 302 and an upper or top surface of the manifold layer 306 directly abuts or contacts an interior surface of the drape 302.

In some embodiments, the drape 302 includes a first opening 310 and a second opening 312 with which a coupler assembly 320 is operably coupled. In some embodiments the coupler assembly 320 is configured to draw a negative pressure within the dressing by fluidly coupling with the inner volume of the drape 302 and skin interface layer 304 through the first opening 210. In some embodiments, the coupler assembly 320 includes a coupler 322, a connector 324, and a first tubular member 326 and a second tubular member 328 (e.g., tubes, conduits, pipes, lines, tubing 108, tubing 110, etc.). In some embodiments, the coupler 322 is configured to fluidly couple the inner volume of the wound dressing 112 with the first tubular member 326 via the first opening 310 for drawing a negative pressure at the wound dressing 112. Specifically, the first tubular member 326 can be fluidly coupled with a NPWT device (e.g., therapy device 102) that includes a pump for drawing a negative pressure at the inner volume of the wound dressing 112. In some embodiments, the first tubular member 326 and second tubular member 328 define fluid flow paths. In some embodiments, the first tubular member 326 is a flexible member. In some embodiments, the first tubular member 326 is an elongated member with a hollow center for drawing a negative pressure at the inner volume of the wound dressing 112 and for drawing exuded wound fluid from the inner volume of the wound dressing 112.

In some embodiments, a leak occurs between the periwound tissue 116 and skin interface layer 304 due to poor adhesion or contact between periwound tissue 116 and skin interface layer 304 (e.g., body hair and/or skin folds may inhibit a reliable seal between skin interface layer 304 and periwound tissue 116). In some embodiments, a leak occurs between the skin interface layer 304 and drape layer 302 due to drape layer 302 being mislaid on skin interface layer 304. For example, drape layer 302 may be mislaid on skin interface layer 304 when drape layer includes creases, folds, or wrinkles that allow air to enter the wound dressing 112. In some embodiments, a leak occurs due to one or more holes in drape layer 302 which may be inadvertently formed during the application of the wound dressing 112 to the wound 114. In some embodiments, a leak occurs in wound dressing 112 due to manufacturing defects. In some embodiments, leaks occur in wound dressing 112 due to improper application (e.g., assembly, placement, etc.) of wound dressing 112. In some embodiments, a leak occurs near first opening 310 and/or second opening 312 due to a poor seal between drape layer 302 and coupler assembly 320. In some embodiments, a leak occurs due to the periwound tissue 116 shifting during movement. For example, a patient may move (e.g., stand, sit, lay down, roll over, etc.) and the wound dressing may become torn, punctured, disengaged, unsealed, etc. which creates a leak in wound dressing 112. A person having ordinary skill in the art will appreciate that additional leak locations and leak causes are possible, and the examples of leak locations and leak causes provided above are for illustration only.

### Wound Detection Using Electronic Stethoscope

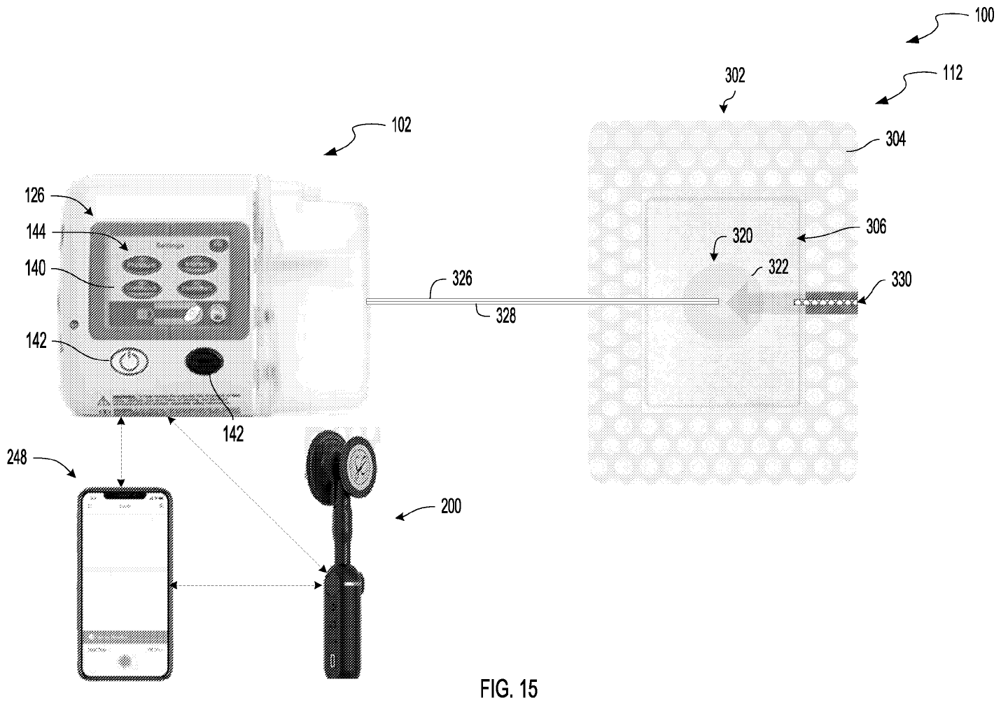

Referring now to FIGS. 15-19, wound dressing 112 is shown with a leak 330. As shown, the leak extends from an outer edge of the skin interface layer 304 and the drape layer 302 to the manifold layer 306 underneath the drape layer 302. In some embodiments, the leak 330 may be a small or narrow break in skin interface layer 304. In some embodiments, leak 330 may generate a sound when air flows through the leak 330. In some embodiments, the sound produced by leak 330 is dependent on at least one of the pressure differential between the ambient environment and the negative pressure drawn at the wound, the geometry of the leak passage (e.g., flow passage), the leak inlet geometry, the leak outlet geometry, the characteristics of the material surrounding the leak (e.g., thickness, rigidity, surface roughness, etc.), air composition (e.g., humidity, pressure, etc.) and still other applicable variables. In some embodiments, an approximate size of a leak may be determined by analyzing the sound (e.g., frequency, amplitude, etc.) produced by the leak 330. In some embodiments, the leak 330 may produce a sound having a frequency within a human perceptible frequency range (e.g., approximately 20 Hz to 20 kHz). In some embodiments, the leak 330 may emit a sound at a frequency above a human perceptible frequency range (e.g., above the range of approximately 20 Hz to 20 kHz). In such embodiments, electronic stethoscope 200 may be configured to detect and isolate a band of frequencies (e.g., 20 Hz to 40 kHz, 10 Hz to 80 kHz, etc.) to facilitate a user detecting a leak outside of the human perceptible frequency range.

Referring particularly to FIG. 15, the NPWT system 100 is shown, according to some embodiments. As shown, the leak 330 is detected by therapy device 102 and the graphical user interface 144 is presented on display 140. In some embodiments, graphical user interface 144 may present the graphical user interface 144 as shown in FIG. 8. In some embodiments, graphical user interface 144 may display instruction window 182 which may present instructions for a user to pair an electronic stethoscope 200 with a user device 248. As shown, the electronic stethoscope 200 is paired (e.g., communicably coupled, communicably connected, etc.) with the user device 248 and the therapy device 102 by connections 202. In some embodiments, the electronic stethoscope 200 may be directly connected with the therapy device 102. In some embodiments, the electronic stethoscope 200 may be directly connected with the user device 248. In some embodiments, the user device 248 may be paired with the electronic stethoscope 200 and may not be paired with the therapy device 102.

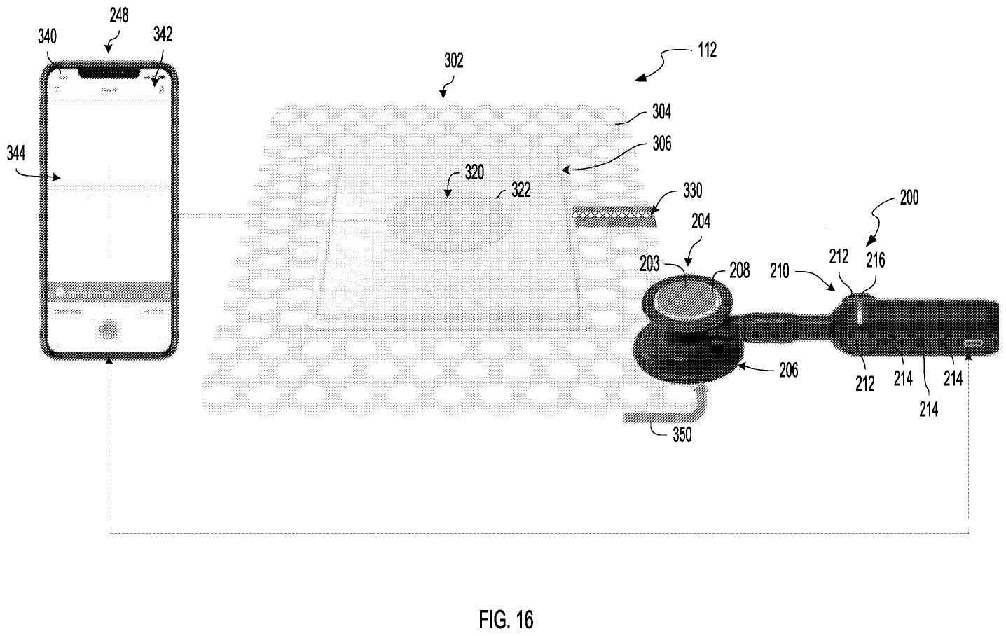

Referring particularly to FIG. 16, electronic stethoscope 200 is paired to user device 248. In some embodiments, user device 248 includes a display 340. In some embodiments, display 340 is the same as or similar to display 140. In some embodiments, user device 248 may present a graphical user interface 342 which may include any feature described with respect to the graphical user interface 144 shown in FIGS. 7-13. Likewise, in some embodiments, the graphical user interface 144 may include any feature described with respect to graphical user interface 342. As shown, graphical user interface 342 is displaying a sound visualization area 344. In some embodiments, sound visualization area 344 may display a spectrogram (e.g., sonograph, voicegram, etc.) which may be a visual representation of the spectrum of frequency signals detected by electronic stethoscope 200 over time.

In some embodiments, the electronic stethoscope 200 a user is instructed (e.g., via instruction window 182) to move bell 204 around wound dressing 112 in a pattern or specific path (e.g., a spiral search method path, a grid search method path, a strip search method path, a line search method path, a quadrant or zone search method path, etc.). In some embodiments, graphical user interface 144 and/or graphical user interface 342 may present an instruction window 182 which may instruct a user to perform the search pattern or specific search path. In some embodiments, instruction window 182 instructs a user to check the periphery of wound dressing 112 for leaks (e.g., leak 330). As shown, electronic stethoscope 200 is being moved in direction 350 around the periphery of the wound dressing 112 to check for leaks.

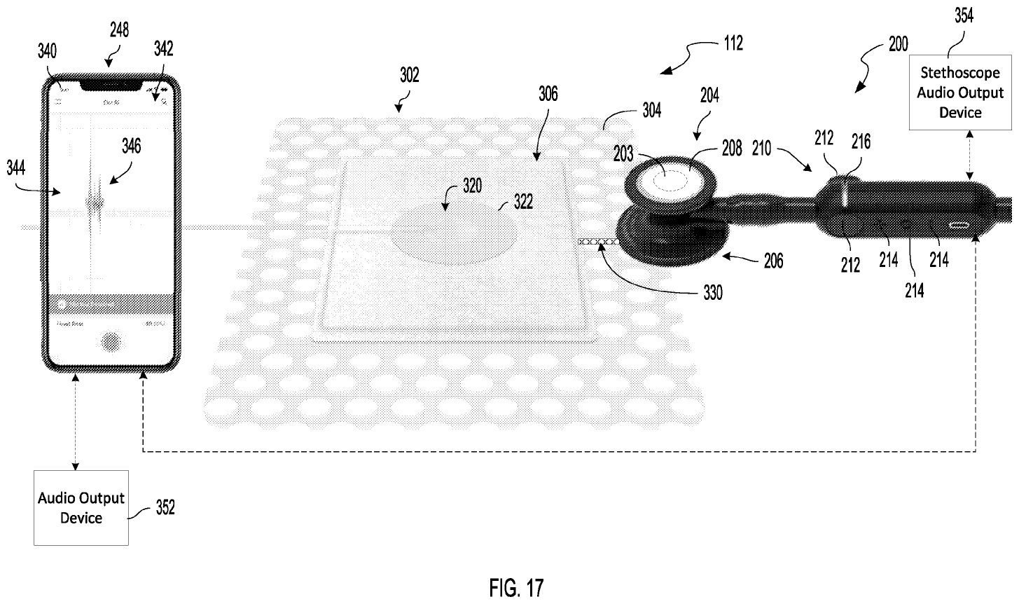

Referring particularly to FIG. 17, the electronic stethoscope 200 has detected the leak 330 while scanning the wound dressing 112. Leak frequency signals 346 are visually presented in sound visualization area 344, according to some embodiments. In some embodiments, leak frequency signals 346 are output (e.g., reproduced) in an auditory format using an audio output device 352 (e.g., speaker, headset, headphones, earbuds, etc.). In some embodiments, an auditory signal representative of leak frequency signals 346 is output (e.g., a beep, a tone, etc.) using an audio output device 352. As shown, audio output device 352 is wirelessly connected to user device 248. In some embodiments, audio output device 352 is hardwired to user device 248, or may be within user device 248 (e.g., a built-in speaker). In some embodiments, user device 248 includes a first audio output device 352 within the user device 248, and a second audio output device 352 (e.g., headphones, earbuds, headset, etc.) communicably connected (e.g., wirelessly connected, etc.) with user device 248. In some embodiments, sound is output from audio output device 352 and leak frequency signals 346 are represented in sound visualization area 344.

In some embodiments, electronic stethoscope 200 may be communicably connected to a stethoscope audio output device 354. For example, the stethoscope audio output device 354 may be a headset, headphones, earbuds, speaker, or other suitable audio output device. As shown, the stethoscope audio output device 354 is wirelessly connected to the electronic stethoscope 200. In some embodiments, the stethoscope audio output device 354 may be hardwired or built into the electronic stethoscope 200. In some embodiments, the audio output device 352 may be used alone or in combination with the stethoscope audio output device 354. For example, the stethoscope audio output device 354 may output leak frequency signals 346 and audio output device 352 may produce a representative auditory signal (e.g., beep, alarm, etc.) to indicate that a leak has been detected by the electronic stethoscope 200. In some embodiments, at least one of audio output device 352 and stethoscope audio output device 354 may be the same as or similar to frequency signal output device 240. Additionally, in some embodiments, indicator 216 may indicate that a leak has been detected by electronic stethoscope 200 by flashing, changing colors, etc. In this way, a combination of auditory signals and visual signals may be produced to alert a user that the leak 330 has been detected by the electronic stethoscope 200 based on frequency signals emitted from the leak 330 and detected by electronic stethoscope 200, according to some embodiments.

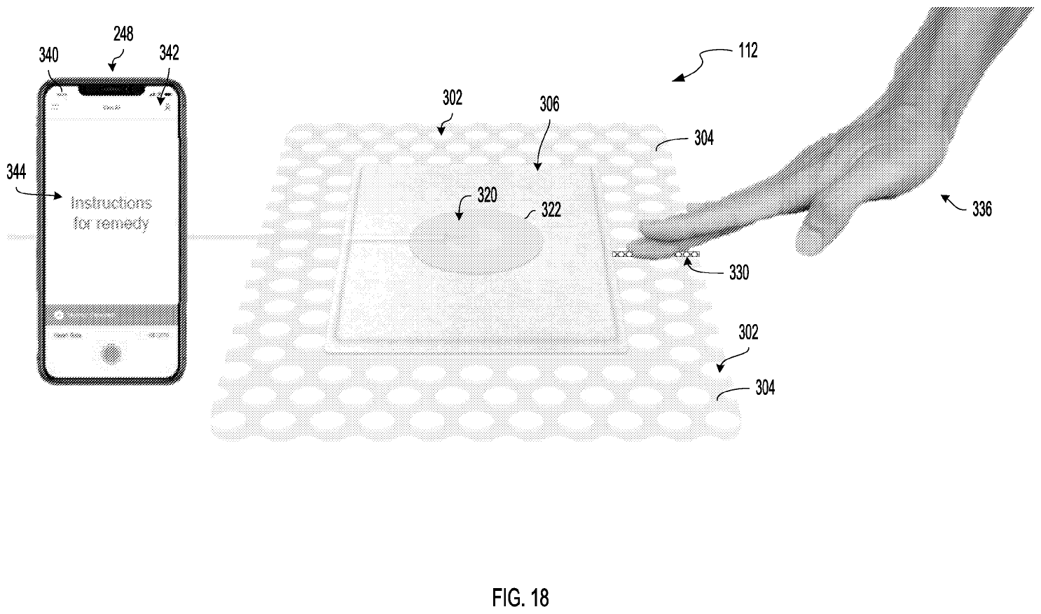

Referring particularly to FIG. 18, a user 336 is pressing (e.g., gently pressing) on the leak 330 to verify the location of the leak 330 detected by electronic stethoscope 200. In some embodiments, the graphical user interface 144 may display seal check leak bar 172, and pressing on the leak 330 may cause the seal check leak bar 172 to display a change in the detected leak rate. In some embodiments, the graphical user interface 342 may present instructions for repairing the leak 330 similar to or the same as the instructions presented on graphical user interface 144. In some embodiments, the instructions for repairing the leak involve steps for repairing the leak 330. For example, the instructions may involve a user preparing (e.g., cleaning, smoothing, drying, etc.) the surfaces surrounding the leak 330 and applying an adhesive patch (e.g., adhesive covering, covering, etc.) which seals over the leak 330. In some embodiments, excess wound dressing 112 materials (e.g., excess drape layer 302) may be used to repair the leak, alone or in combination with other materials (e.g., adhesives, excess skin interface layer 304, tape, etc.). In some embodiments, the instructions for repairing the leak may indicate that a replacement or reapplication of the wound dressing 112 to the wound 114 is required (e.g., when the leak rate is severe).

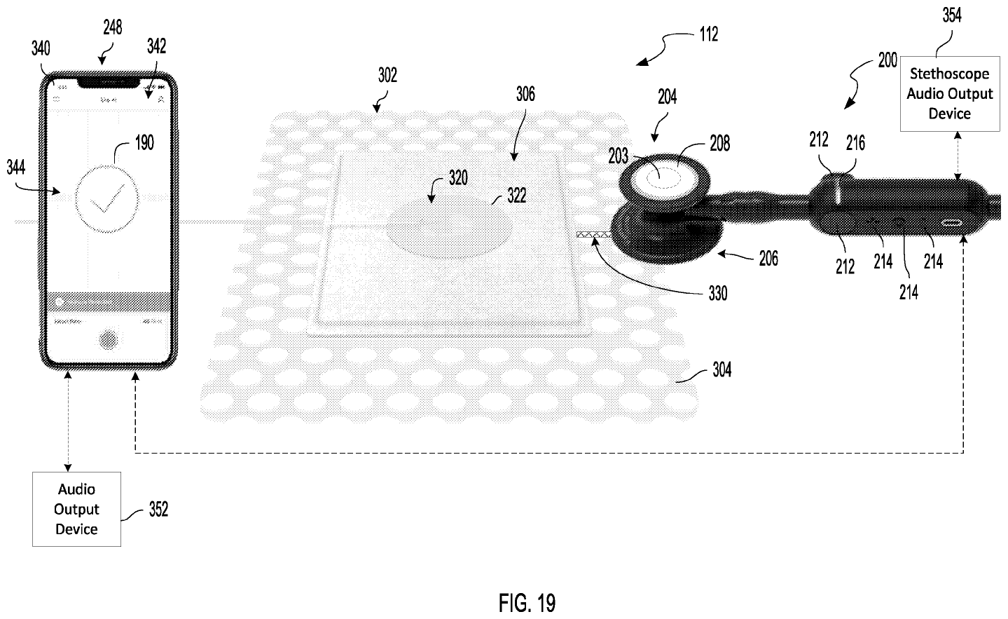

Referring particularly to FIG. 19, the user 336 has performed the instructions presented on the graphical user interface 144 and/or graphical user interface 342 to repair leak 330, and electronic stethoscope 200 has been placed nearby the location of the repair to leak 330 to check the effectiveness of the repair. In some embodiments, the repair may include a new leak 330 which may require an additional repair. In some embodiments, a user may repair a first leak 330 but a second leak 330 may be present elsewhere on wound dressing 112. In such embodiments, a user may be instructed to scan the wound dressing 112 using the electronic stethoscope 200 using the method used for determining the location of the first leak 330.

In some embodiments, a success symbol 190 is presented on the graphical user interface 144 and/or graphical user interface 342 when a repair has been applied successfully (e.g., no leak sounds detected by electronic stethoscope 200 and the leak rate is below leak threshold 174, etc.).

It is important to note that in some embodiments the NPWT system 100 only includes a single display (e.g., a display 140, a display 340, a display 244). In such embodiments, the graphical user interfaces 144, 342 may be displayed on the single display. For example, the user device 248 may be communicably coupled with the therapy device 102 and the graphical user interface 144 may be displayed on display 340. In some embodiments, one or more features of graphical user interface 342 (e.g., sound visualization area 344) are incorporated into graphical user interface 144. Likewise, in some embodiments, one or more features of graphical user interface 144 (e.g., system status portion 150, communications signal strength indicator 152, therapy status button 166, etc.) may be incorporated into graphical user interface 342.

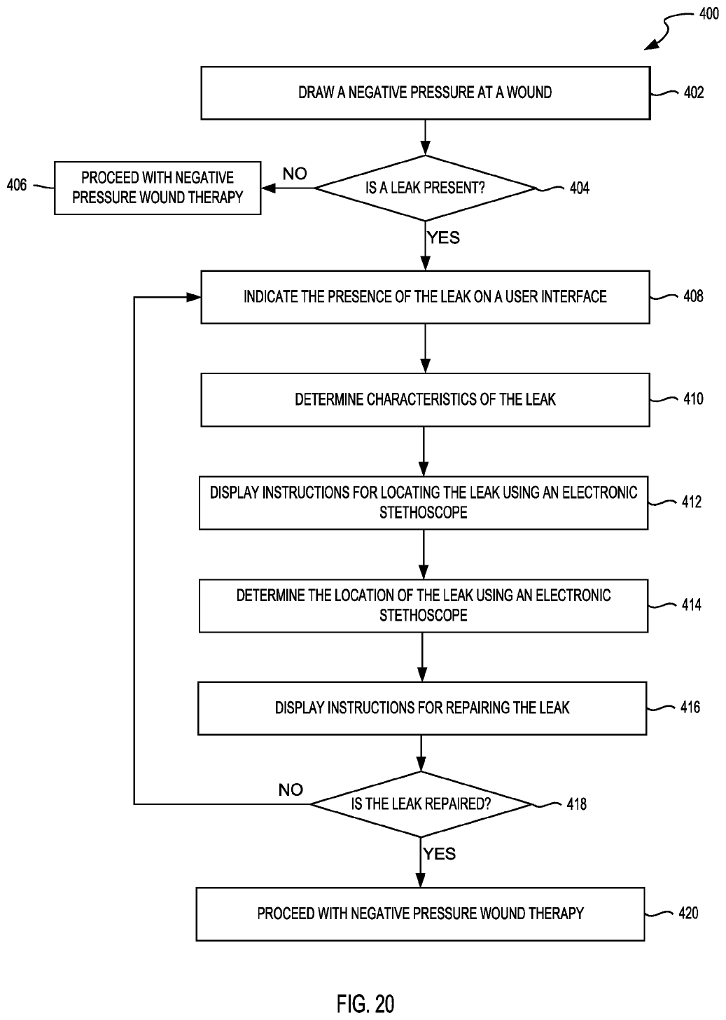

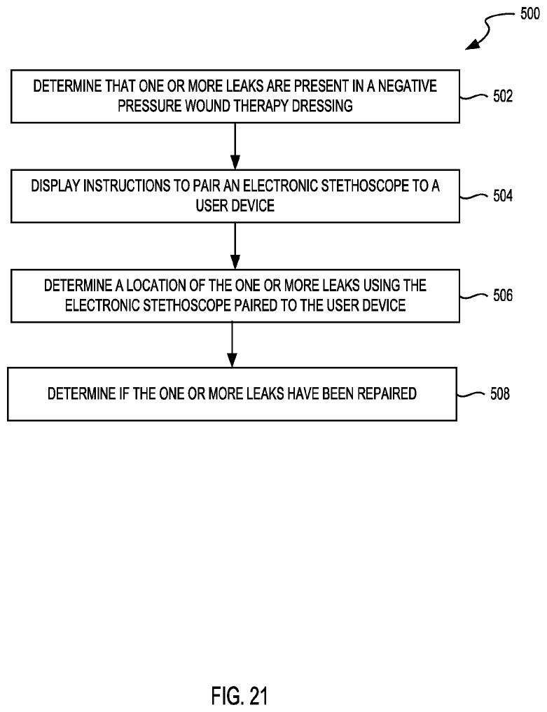

Referring particularly to FIG. 20, a process 400 for detecting and repairing a NPWT dressing leak is shown, according to some embodiments. In some embodiments, process 400 includes steps 402-420 and can be performed using the therapy device 102, wound dressing 112, user device 248, and electronic stethoscope 200 as shown in any of the configurations or embodiments shown in FIGS. 1-19 or any combination thereof. In some embodiments, process 400 is performed to facilitate healing of a wound.

Process 400 includes drawing a negative pressure at a wound (step 402), according to some embodiments. In some embodiments, drawing a negative pressure at a wound involves generating a negative pressure at the sealed inner volume of the wound defined by wound dressing 112. In some embodiments, the NPWT device (e.g., therapy device 102) draws a negative pressure at the wound dressing 112 via the coupler (e.g., coupler assembly 300). In some embodiments, step 402 includes operating the pneumatic pump 120 to draw the negative pressure at the dressing via a tubular member (e.g., a dedicated tubular member 326) and the coupler (e.g., coupler assembly 300). In some embodiments, the NPWT device is activated and the pneumatic pump 120 draws a negative pressure at the sealed inner volume of the wound dressing 112. In some embodiments, the pneumatic pump 120 draws a static or a dynamic negative pressure at the wound. In some embodiments, oscillating the negative pressure changes the surface topology of the wound to facilitate an enhanced (e.g., improved) NPWT treatment.

Process 400 includes determining if a leak is present (step 404), according to some embodiments. In some embodiments, step 404 includes determining a leak rate within the negative pressure wound circuit. For example, therapy device 102 may monitor a duty cycle of pneumatic pump 120 and/or may monitor the pressure sensors 113, 130 to determine if an unexpected leak is present in the negative pressure circuit. In some embodiments, the leak rate determined by the therapy device 102 is compared to a predetermined value (e.g., leak threshold 174, an alarm threshold, etc.). In some embodiments, if no leaks are present in the negative pressure circuit, process 400 continues with proceeding with negative pressure wound therapy treatment (step 406). In some embodiments, if leaks are present in the negative pressure circuit (e.g., an unexpected leak rate is detected), process 400 indicates the presence of the leak on a user interface (step 408).

Process 400 includes indicating the presence of the leak on a user interface (step 408). In some embodiments, the determined leak rate or an indication of the determined leak rate is displayed on a graphical user interface (e.g., graphical user interface 144). For example, the leak rate may be indicated by the seal check indication ring 170 and or seal check leak bar 172. In some embodiments, an indication of the leak rate may be presented in an auditory format (e.g., an alarm). In some embodiments, the leak rate may be indicated by at least one light emitting device (e.g., light emitting diode). For example, a multicolored light emitting device may be configured to flash, change intensity, change color, etc. based on the leak rate determined by the therapy device 102.

Process 400 includes determining characteristics of the leak (step 410). In some embodiments, the characteristics of the leak include determining the leak rate attributed to the leak. For example the leak rate attributed to the leak may be calculated as the difference between the unexpected leak rate (e.g., total leak rate) and the expected leak rate (e.g., the expected rate of loss of pressure in the negative pressure circuit during NPWT treatment).

Process 400 includes displaying instructions for locating a leak using an electronic stethoscope (step 412), according to some embodiments. In some embodiments, the instructions for locating a leak using an electronic stethoscope include instructions for pairing (e.g., communicably connecting) the electronic stethoscope 200 to at least one of the therapy device 102 and the user device 248. In some embodiments, the instructions for pairing the electronic stethoscope 200 may include pairing the electronic stethoscope 200 to the user device 248, and pairing the user device 248 to the therapy device 102. In some embodiments, the instructions for locating the leak using the electronic stethoscope 200 are displayed in the instruction window 182.

Process 400 includes determining the location of the leak using an electronic stethoscope (step 414), according to some embodiments. In some embodiments, step 414 includes instructing a user to move the electronic stethoscope 200 around the wound dressing 112 to determine the location of a leak (e.g., leak 330). For example, a user may be instructed to move the electronic stethoscope 200 around the wound dressing 112 as described with respect to FIG. 16. In some embodiments, the leak is located when the electronic stethoscope 200 detects frequency signals (e.g., sounds) determined to be correlated with a leak 330. In some embodiments, the leak may be indicated to a user as described with respect to FIG. 17.

In some embodiments, the therapy device 102 may be configured to modify the pump duty of the pneumatic pump 120 to facilitate identifying leaks with the electronic stethoscope 200 (e.g., by reducing or stopping pump noise, by drawing the negative pressure to a different negative pressure to intensify or lessen the sounds emitted by the leak, etc.). In some embodiments, therapy device 102 is configured to draw the wound to a known (e.g., specific, predetermined, etc.) negative pressure and the negative pressure pump (e.g., pneumatic pump 120) is turned off to reduce the background noise for characterization of the leak by the electronic stethoscope 200.

In some embodiments, the electronic stethoscope 200 is configured to perform active noise canceling to cancel (e.g., attenuate, destroy, remove, filter, etc.) ambient noise (e.g., pneumatic pump 120 noise, ambient HVAC noise, etc.) from the sounds transduced by the electronic stethoscope 200. In some embodiments, the filtered (e.g., processed, etc.) sound is correlated against sound profiles stored in the frequency signal database 228 to determine the size and/or scale of the leak. In such embodiments, the electronic stethoscope 200 may determine if the unexpected leak rate is attributed to the leak detected by the electronic stethoscope 200. For example, the electronic stethoscope 200 may determine that the leak detected by the electronic stethoscope 200 only accounts for a portion (e.g., half) of the unexpected leak rate determined by the therapy device 102. In such example, the therapy device 102 and/or the electronic stethoscope 200 may indicate that a second leak is present beyond the immediate leak detected by the electronic stethoscope 200. In some embodiments, the electronic stethoscope 200 may be configured to determine the leak rate in standard units (e.g., cc/min, etc.) or scale values (e.g., small, medium, large, etc.).

In some embodiments, wound dressing 112 is configured to indicate a location of the leak using electronic devices installed in the wound dressing 112. For example, one or more frequency signal transducers (e.g., microphones, vibration sensors, etc.) and one or more light emitting devices corresponding to the one or more microphones may be located around wound dressing 112. In such example, the microphone and the light emitting device may be configured to detect a leak (e.g., via the microphone) and indicate the presence of the leak (e.g., via the light emitting device) within the detectable range of the microphone. The indication may facilitate a user being directed to scan with the electronic stethoscope 200 the portion or section of the wound dressing 112 where a leak is detected (e.g., by the microphone associated with the wound dressing 112). In some embodiments, the microphone, light emitting device, and associated circuitry (e.g., processor, memory, ASIC, etc.) are powered by a local power source (e.g., battery). In some embodiments, the microphone, light emitting device, and associated circuitry are powered using wireless power transfer (e.g., wireless power transmission, etc.). In some embodiments, wound dressing 112 is configured for ultra-wideband (UWB) communication. In such embodiments, power may be transmitted from a device placed nearby the wound dressing 112 to the microphone, light emitting device, and associated circuitry. In some embodiments, the microphone, light emitting device, and associated circuitry associated with wound dressing 112 are placed above and/or below drape layer 302. In some embodiments, the microphone, light emitting device, and associated circuitry are coupled (e.g., glued, adhered, etc.) to the exterior of drape layer 302 and/or the periwound tissue 116.

Process 400 includes displaying instructions for repairing the leak (step 416). In some embodiments, the instructions for repairing the leak may include a user preparing (e.g., cleaning, smoothing, drying, etc.) the surfaces surrounding the leak 330 and applying an adhesive patch (e.g., adhesive covering, covering, tape, etc.) which seals over the leak 330. In some embodiments, excess wound dressing 112 materials (e.g., excess drape layer 302) may be used to repair the leak, alone or in combination with other materials (e.g., adhesives, skin interface layer 304, etc.). In some embodiments, the instructions for repairing the leak may indicate that a replacement or reapplication of the wound dressing 112 to the wound 114 is required.

Process 400 includes determining if the leak has been repaired (step 418). In some embodiments, the graphical user interface 144 displays instructions for checking the repair using the electronic stethoscope 200. For example, the instructions may include placing the electronic stethoscope 200 near the location of the repair to determine if new leaks are present (e.g., by detecting new frequency signals indicative of a leak). If a new leak is present, or the leak rate remains above the expected leak rate or the leak threshold 174, process 400 may return to step 408. In some embodiments, the therapy device 102 determines that the leak rate is within a tolerance of the expected leak rate (e.g., +10%) and/or less than the leak threshold 174 to determine if the leak has been repaired (e.g., by ending an alarm, turning off a light emitting device, displaying success symbol 190, etc.). In such example, process 400 may continue with negative pressure wound therapy (step 420).

Referring particularly to FIG. 20, a process 500 for determining the location of a leak is shown, according to some embodiments. In some embodiments, process 500 includes steps 502-508 and can be performed using the therapy device 102, wound dressing 112, user device 248, and electronic stethoscope 200 as shown in any of the configurations or embodiments shown in FIGS. 1-19 or any combination thereof. In some embodiments, process 500 is performed to facilitate healing of a wound.

Process 500 includes determining that a leak is present in a negative pressure wound therapy (NPWT) dressing (step 502), according to some embodiments. In some embodiments, step 502 includes drawing a negative pressure at a wound and monitoring the pressure in the negative pressure circuit as described with respect to step 402 and step 404 above.

Process 500 includes displaying instructions to pair an electronic stethoscope to a user device (step 504), according to some embodiments. In some embodiments, the user device is the user device 248. In some embodiments, step 504 includes instructions to pair (e.g., communicably couple, communicably connect, etc.) the user device 248 to the electronic stethoscope 200 and/or the therapy device 102. In some embodiments the instructions for pairing the electronic stethoscope 200 to the user device 248 are displayed in the instruction window 182.

Process 500 includes determining the location of the one or more leaks using the electronic stethoscope paired to the user device (step 506), according to some embodiments. In some embodiments, a user may move the electronic stethoscope 200 around the wound dressing 112 according to instructions displayed on user device 248 (e.g., in an instruction window 182). In some embodiments, a user may toggle between an amplified mode (e.g., a noise canceling mode, a digitally filtered mode, etc.) and a standard mode (e.g., an unfiltered mode) using toggle switch 212. In some embodiments, the user device 248 may receive and display (e.g., on graphical user interface 342) information from both the therapy device 102 (e.g., status information, leak rate, etc.) and the electronic stethoscope 200 (e.g., connectivity information, detected frequency signals, etc.) to facilitate a user determining the location of the leak. In some embodiments, the user device 248 communicates information from electronic stethoscope 200 to the therapy device 102. Likewise, in some embodiments, the user device 248 communicates information from the therapy device 102 to the electronic stethoscope 200. In some embodiments, the user device 248 is only communicably connected with electronic stethoscope 200 and information (e.g., instructions, user prompts, etc.) is stored on the user device 248 (e.g., in an on-board memory device) and/or on electronic stethoscope 200 (e.g., in memory 224) for generating the graphical user interface 342.

Process 500 includes determining if the one or more leaks have been repaired (step 508), according to some embodiments. In some embodiments, the one or more leaks may be repaired by a user, and the therapy device 102 may be configured to determine if the leak rate is below a threshold value (e.g., leak threshold 174). In some embodiments, the electronic stethoscope 200 is used to detect the presence of an additional leak or a persisting leak. For example, the electronic stethoscope 200 may detect additional frequency signals emitted by additional leaks in either the repair or the wound dressing 112, according to some embodiments.

In some embodiments, the electronic stethoscope 200 is configured to provide feedback for performing a seal check without being paired to user device 248 and/or therapy device 102. In some embodiments, electronic stethoscope 200 includes a built-in interface (e.g., stethoscope user interface 210) and one or more light emitting devices (e.g., light emitting diodes, indicator 216, etc.), sound output devices (e.g., frequency signal output device 240, speakers, piezoelectric device, etc.) and/or displays (e.g., liquid-crystal display, LED display, OLED display, electrophoretic display, etc.) to provide feedback to a user for performing the seal check. For example, a user may receive visual feedback (e.g., from indicator 216) and auditory feedback (e.g., frequency signals, an indication of frequency signals, etc.) when the frequency signal transducer 203 of the electronic stethoscope 200 detects frequency signals associated with a leak. In some embodiments, a user may enter a seal check mode on the electronic stethoscope 200 by interacting with the stethoscope user interface 210 (e.g., buttons 214). In such embodiments, the electronic stethoscope 200 may be used in steps 502, 506, and 508 without being paired to the therapy device 102 and/or the user device 248.

### Therapy Device Controller

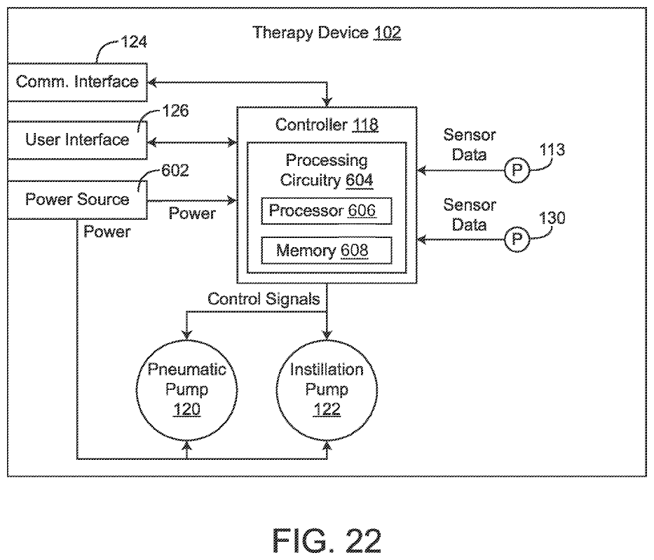

Referring particularly to FIG. 22, a block diagram of the therapy device 102 configured to operate the pneumatic pump 120 (e.g., for NPWT) and the instillation pump 122 (e.g., for providing instillation fluid, a saline solution, a solution including a photosensitizing agent, etc.) is shown, according to some embodiments. In some embodiments, the power source 602 is configured to provide electrical power to any of the controller 118, the pneumatic pump 120, and the instillation pump 122. In some embodiments, the controller 118 is also configured to obtain sensor data from the pressure sensors 113 and 130. In some embodiments, the controller 118 is configured to generate control signals for the pneumatic pump 120, and/or the instillation pump 122, to provide NPWT and/or phototherapy to a patient's wound.

The controller 118 is shown to include processing circuitry 604 including a processor 606 and memory 608. The processing circuitry 604 can be communicably connected to the communications interface 124 such that the processing circuitry 604 and the various components thereof can send and receive data via the communications interface 124. The processor 606 can be implemented as a general purpose processor, an application specific integrated circuit (ASIC), one or more field programmable gate arrays (FPGAs), a group of processing components, or other suitable electronic processing components.

The memory 608 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. The memory 608 can be or include volatile memory or non-volatile memory. The memory 608 can include database components, object code components, script components, or any other type of information structure for supporting the various activities and information structures described in the present application. According to some embodiments, the memory 608 is communicably connected to the processor 606 via the processing circuitry 604 and includes computer code for executing (e.g., by the processing circuitry 604 and/or the processor 606) one or more processes described herein.

### ALTERNATIVE EMBODIMENTS

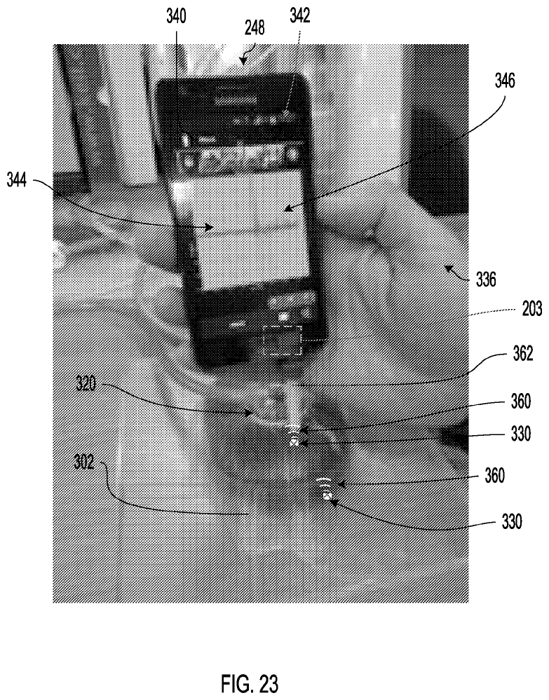

Referring now to FIG. 23, the user 336 is using the user device 248 to check drape layer 302 for leaks 330 (e.g., pinhole leaks). The leaks 330 are producing (e.g., emitting) leak frequency signals 360 (e.g., leak sounds, sound, soundwaves, acoustic signals, etc.). A Hemholtz Resonator 362 is coupled to the user device and is augmenting the vibratory motion of the leak frequency signals 360 (e.g., soundwaves). The augmented leak frequency signals are being detected by the frequency signal transducer 203. The user device 248 is displaying a visual representation of the leak frequency signals 360 (e.g., leak frequency signals 346) in the sound visualization area 344.

In some embodiments, a frequency signal transducer 203 is native to the user device 248. In such embodiments, the user device 248 may include some or all of the elements described with respect to the electronic stethoscope 200 shown in FIG. 6. For example, the user device 248 may include the audio manager 226, frequency signal database 228, and communications module 246. In some embodiments, the frequency signal transducer 203 is associated (e.g., built into, native to, coupled, communicably coupled, etc.) with the user device 248. In some embodiments, detecting the leak (e.g., step 414) involves a user 336 moving the user device 248 around the wound dressing 112.

Still referring to FIG. 23, the position of the leak 330 is determined by detecting an increase in amplitude of frequency signals (e.g., sounds) detected by the frequency signal transducer 203 when in a close proximity to the leak 330. The Hemholtz Resonator 362 is improving (e.g., enhancing, etc.) the effective sensitivity of the frequency signal transducer 203 by augmenting the vibratory motion of the leak frequency signals 360 (e.g., sounds).

As described herein, electronic stethoscope 200 may be embodied as any listening device capable of detecting frequency signals (e.g., leak frequency signals 360) emitted by a leak (e.g., leak 330). For example, electronic stethoscope 200 may resemble a wand (e.g., stick, elongated member, etc.) and may have a frequency signal transducer 203 coupled to an end. In such example, the frequency signal transducing end (e.g., the end having the frequency signal transducer 203) may be moved around more quickly and easily than in embodiments that require the entire electronic stethoscope 200 to be moved around the wound dressing 112.

### CONFIGURATION OF EXEMPLARY EMBODIMENTS

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A method for checking a seal of a negative pressure wound therapy (NPWT) dressing, the method comprising:
detecting a presence of a leak in a NPWT dressing using a NPWT device;
displaying, via a user interface, instructions to identify the leak using an electronic stethoscope; and
determining a location of the leak in the NPWT dressing using the electronic stethoscope.

2. The method of claim 1, wherein the instructions to identify the leak using an electronic stethoscope comprise displaying a plurality of steps for performing by a user, and wherein the plurality of steps comprise moving a transducer of the electronic stethoscope around the NPWT dressing.

3. The method of claim 2, wherein the plurality of steps further comprise steps for pairing the electronic stethoscope to at least one of the NPWT device or a user device.

4. The method of claim 3, wherein the user device is in communication with the NPWT device.

5. The method of claim 1, further comprising measuring a characteristic of the leak using the electronic stethoscope.

6. The method of claim 5, further comprising determining a flow rate of the leak based on the characteristic measured by the electronic stethoscope.

7. The method of claim 5, further comprising displaying, via graphical user interface, the characteristic of the leak.

8. The method of claim 5, further comprising indicating the characteristic of the leak via at least one of a light emitting device and a sound emitting device.

9. The method of claim 1, further comprising determining a characteristic of the leak based on a static negative pressure applied to the NPWT dressing and based on a signal generated by a transducer of the electronic stethoscope.

10. The method of claim 9, wherein a negative pressure pump of the NPWT device is disabled while the signal generated by the transducer of the electronic stethoscope is obtained.

11. The method of claim 1, wherein the NPWT device comprises the user interface, and wherein the user interface comprises a graphical user interface.

12. The method of claim 1, wherein the electronic stethoscope is configured for active noise cancelation.

13. The method of claim 1, wherein the electronic stethoscope comprises a Helmholtz resonator configured to augment vibratory motion of sound waves generated by the leak.

14. A method of detecting and repairing a negative pressure wound therapy (NPWT) dressing leak, the method comprising:
detecting a presence of one or more leaks in a NPWT dressing using a NPWT device comprising a first user interface, a negative pressure pump, a canister, and processing circuitry;
displaying, via the first user interface, instructions to locate the one or more leaks using an electronic stethoscope;
determining one or more locations of the one or more leaks on the NPWT dressing using the electronic stethoscope; and
displaying, via the first user interface, instructions for repairing the one or more leaks at the one or more locations determined using the electronic stethoscope.

15. The method of claim 14, wherein the instructions for repairing the one or more leaks comprise applying additional dressing to the one or more locations of the one or more leaks.

16. The method of claim 14, wherein the electronic stethoscope is configured to selectively filter frequency signals detected by a transducer of the electronic stethoscope.

17. An advanced seal check system for negative pressure wound therapy (NPWT), the system comprising:
a dressing comprising:
a manifold layer for NPWT;
a drape layer covering the manifold layer, the drape layer configured to be sealingly coupled with skin surrounding the wound and defining a sealed inner volume of the dressing, wherein the drape layer has an opening for drawing a negative pressure at the sealed inner volume of the dressing;
a NPWT device comprising:
a negative pressure pump configured to generate the negative pressure at the sealed inner volume of the dressing;
a canister configured to collect fluid secreted by the wound during NPWT;
a user interface comprising a display; and
a controller comprising processing circuitry configured to:
detect a leak in the dressing;
determine a characteristic of the leak in the dressing; and
display, via a graphical user interface on the display, the characteristic of the leak in the dressing; and
an electronic stethoscope configured to be communicably coupled with at least one of the NPWT device and a user device, the electronic stethoscope comprising a transducer for detecting frequency signals generated by the leak in the dressing.

18. The advanced seal check system of claim 17, wherein the processing circuitry is further configured to display, via the graphical user interface on the display, instructions to locate the leak using the electronic stethoscope.

19. (canceled)

20. The advanced seal check system of claim 17, wherein the electronic stethoscope is configured to digitally filter the frequency signals detected by the transducer to isolate a second frequency signals emitted by the leak in the dressing.

21. A method for checking a seal of a negative pressure wound therapy (NPWT) dressing, the method comprising:
detecting a leak in a NPWT dressing using a NPWT device;
displaying, via a user interface, instructions to identify the leak using a listening device; and
detecting a location of the leak in the NPWT dressing based on a sound detected by the listening device.

22. (canceled)

23. (canceled)

24. (canceled)

25. (canceled)

26. (canceled)
